(19) United States
(12) Patent Application Publication
(10) Pub. No.: US 2020/0080058 A1
(43) Pub. Date: Mar. 12, 2020

(54) METHODS OF IMPROVING HEMATOPOIETIC GRAFTS

(71) Applicants: ETABLISSEMENT FRANCAIS DU SANG, LA PLAINE SAINT DENIS (FR); INSERM (INSTITUT NATIONAL DE LA SANTÉ ET DE LA RECHERCHE MÉDICALE), PARIS (FR); UNIVERSITE PARIS-SUD 11, ORSAY (FR); CENTRE NATIONAL DE LA RECHERCHE SCIENTIFIQUE, PARIS (FR); INSTITUT DE RADIOPROTECTION ET DE SÛRETÉ NUCLÉAIRE, FONTENAY AUX ROSES (FR); SORBONNE UNIVERSITÉ, PARIS (FR)

(72) Inventors: LAURENCE GUYONNEAU-HARMAND, VILLEJUIF (FR); CHRISTOPHE DESTERKE, PARIS (FR); THIERRY JAFFREDO, BONDY (FR); ALAIN CHAPEL, YERRES (FR); LOÏC GARCON, PARIS (FR); LUC DOUAY, PARIS (FR)

(21) Appl. No.: 16/495,846

(22) PCT Filed: Mar. 21, 2018
(86) PCT No.: PCT/EP2018/057197

§ 371 Date: Sep. 20, 2019

(30) Foreign Application Priority Data
Mar. 21, 2017 (EP) 17305318.2

(51) Int. Cl. C12N 5/0789; A61K 35/28

(52) U.S. Cl. CPC C12N 5/0647; A61K 35/28; C12N 2501/125; C12N 2501/145; C12N 2501/20; C12N 2501/155; C12N 2501/2306; C12N 2501/2303; C12N 2501/2301; C12N 2501/22; C12N 2501/105; C12N 2506/45; C12N 2501/165

(57) ABSTRACT

The present invention relates to a method of preparing hematopoietic cell graft or enriching a population of cells for hematopoietic stem cells that are capable of long-term multilineage engraftment and self-renewal. It also relates to hematopoietic grafts comprising said hematopoietic stem cells as well as their uses in therapy.

### FIELD OF THE INVENTION

The present invention relates to the field of medicine, in particular to human hematopoietic graft. Specifically, the invention relates to the identification and selection of hematopoietic stem cells that are capable of long-term multilineage engraftment and self-renewal, i.e. hematopoietic stem cells that are suitable to hematopoietic transplantation.

### BACKGROUND OF THE INVENTION

Hematopoietic Stem Cells (HSCs) are the rare cells within human bone marrow (BM) and blood responsible for the life-long curative effects of allogeneic hematopoietic cell transplantation in hematological diseases or following radio/chemotherapy. These cells can be harvested from several sources including BM, mobilized peripheral blood or human umbilical cord blood. Cord blood offers several advantages, namely the reduced need for HLA matching and a decreased risk of graft versus host disease. However despite progress in the manipulation of HSCs, their number remains often insufficient for allogeneic transplantation and the resulting cells displayed reduced multilineage and engraftment potentials compared to freshly isolated HSCs. On the basis of these findings, generating transplantable HSCs from non-hematopoietic sources appears as one of the major goals in regenerative medicine.

A number of protocols were developed using either direct conversion of multiple cell types including cell fusion, reprogramming of differentiated cells through enforced expression of transcription factors, or directed differentiation from human pluripotent stem cells (Wahlster and Daley, Nature cell biology, 2016, 18, 1111-1117). In many cases, the introduction of plasmids encoding oncogenes or utilization of non GMP-grade feeder cells into recipient cells precludes their use for clinical applications. Finally, many of these protocols aim at producing a cell population with a surface phenotype close to bona fide transplantable umbilical cord blood or adult HSCs, a strategy proven to produce cells with a poor engraftment potential.

Consequently, there remains a need for products and methods that improve hematopoietic transplantation efficiency, including enhanced engraftment potential of transplanted cells and improved myeloid replacement.

### SUMMARY OF THE INVENTION

The present invention aims to provide products and methods that improve hematopoietic transplantation efficiency. In particular, the invention provides methods to obtain and select hematopoietic stem cells that are capable of long-term multilineage engraftment and self-renewal in vivo. This invention opens the way to the use of pluripotent stem cells, and in particular induced pluripotent stem cells, as a source of cells for HSC transplantation.

Accordingly, the present invention relates to an in vitro method of preparing hematopoietic cell graft or enriching a population of cells for hematopoietic stem cells that are capable of long-term multilineage engraftment and self-renewal, said method comprising

a) providing a population of cells comprising hematopoietic stem cells, preferably early primitive hematopoietic stem cells, and

b) sorting cells of said population based on the expression of cell surface antigens CD135 and/or CD110, and

c) recovering cells that are CD135+ and/or CD110+.

Preferably, in step b) cells are sorted based on the expression of cell surface antigen CD110, and in step c) recovered cells are CD110+. Optionally, in step b) cells may be further sorted based on the expression of cell surface antigen CD135, and in step c) recovered cells may be CD110+ CD135+.

The method may further comprise, before, after or simultaneously to step b), sorting cells based on the expression of the apelin receptor (APLNR) and recovering cells that are APLNR+.

The population of cells provided in step a) may comprise hematopoietic stem cells obtained from peripheral blood, placental blood, umbilical cord blood, bone marrow, liver and/or spleen and/or may comprise immortalized hematopoietic stem cells.

Alternatively, or additionally, the population of cells provided in step a) may comprise hematopoietic stem cells obtained from in vitro differentiation of pluripotent stem cells, preferably selected from induced pluripotent stem cells or embryonic stem cells, more preferably induced pluripotent stem cells.

In some embodiments, the method may further comprise, before step a), providing pluripotent stem cells, preferably induced pluripotent stem cells inducing embryoid body (EBs) formation,

culturing EBs in a liquid culture medium triggering differentiation of the pluripotent stem cells into the endo-hematopoeitic lineage, and

dissociating EB cells,

thereby obtaining the population of cells provided in step a).

Preferably, the liquid culture medium comprises stem cell factor (SCF), thrombopoietin (TPO), FMS-like tyrosine kinase 3 (FLT3) ligand, bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

Preferably, the pluripotent stem cells are cultured in the liquid culture medium for 14 to 19 days, preferably for 15 to 18 days, more preferably for 17 days.

In another aspect, the present invention also relates to the use of CD135 and/or CD110 as markers of hematopoietic stem cells that are capable of engraftment, and in particular of long-term multilineage engraftment and self-renewal.

In a further aspect, the present invention relates to a hematopoietic cell graft comprising cells and a pharmaceutically acceptable carrier, wherein at least 10% of cells are CD135+ and/or CD110+ hematopoietic stem cells. It also relates to a hematopoietic cell graft prepared according to the method of the invention.

In another aspect, the present invention also relates to a hematopoietic cell graft of the invention for use in the treatment of malignant diseases such as multiple myeloma, non-Hodgkin's lymphoma, Hodgkin's disease, acute myeloid leukemia, acute lymphoblastic leukemia, chronic myeloid leukemia, myelodysplastic syndromes, myeloproliferative disorders, chronic lymphocytic leukemia, juvenile chronic myeloid leukemia, neuroblastoma, ovarian cancer and germ-cell tumors, or non-malignant diseases such as autoimmune disorders, amyloidosis, aplastic anemia, paroxysmal nocturnal hemoglobinuria, Fanconi's anemia, Blackfan-Diamond anemia, thalassemia major, sickle cell anemia, severe combined immunodeficiency, Wiskott-Aldrich syndrome and inborn errors of metabolism.

The hematopoietic stem cell graft may be used in autologous, syngeneic or allogeneic transplantation.

In a further aspect, the present invention also relates to a liquid cell culture medium comprising (i) plasma, serum, platelet lysate and/or serum albumin, and (ii) transferrin or a substitute thereof, insulin or a substitute thereof, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1), preferably a liquid cell culture medium comprising (i) plasma, serum and/or platelet lysate, and (ii) transferrin, insulin, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

In particular, the liquid cell culture medium may comprise

from 10 to 100 ng/mL of SCF, preferably from 10 to 50 ng/mL of SCF;

from 10 to 100 ng/mL of TPO, preferably from 10 to 50 ng/mL of TPO;

from 100 to 500 ng/mL of FLT3-L, preferably from 250 to 350 ng/mL of FLT3-L;

from 10 to 100 ng/mL of BMP4, preferably from 10 to 50 ng/mL of BMP4;

from 50 to 300 ng/mL of VEGF, preferably from 150 to 250 ng/mL of VEGF;

from 10 to 100 ng/mL of IL3, preferably from 20 to 80 ng/mL of IL3;

from 10 to 100 ng/mL of IL6, preferably from 20 to 80 ng/mL of IL6;

from 1 to 20 ng/mL of IL1, preferably from 1 to 10 ng/mL of IL1;

from 10 to 200 ng/mL of GCSF, preferably from 50 to 150 ng/mL of GCSF; and/or

from 10 to 150 ng/mL of IGF1, preferably from 10 to 100 ng/mL of IGF1.

Preferably, the liquid cell culture medium comprises

from 10 to 100 ng/mL of SCF, preferably from 10 to 50 ng/mL of SCF;

from 10 to 100 ng/mL of TPO, preferably from 10 to 50 ng/mL of TPO;

from 10 to 100 ng/mL of FLT3-L, preferably from 10 to 50 ng/mL of FLT3-L;

from 50 to 300 ng/mL of BMP4, preferably from 150 to 250 ng/mL of BMP4;

from 50 to 300 ng/mL of VEGF, preferably from 150 to 250 ng/mL of VEGF;

from 10 to 100 ng/mL of IL3, preferably from 20 to 80 ng/mL of IL3;

from 10 to 100 ng/mL of IL6, preferably from 20 to 80 ng/mL of IL6;

from 1 to 20 ng/mL of IL1, preferably from 1 to 10 ng/mL of IL1;

from 10 to 200 ng/mL of GCSF, preferably from 50 to 150 ng/mL of GCSF; and/or

from 1 to 20 ng/mL of IGF1, preferably from 1 to 10 ng/mL of IGF1.

The liquid culture medium may further comprise (i) plasma, serum, platelet lysate and/or serum albumin, preferably plasma, serum and/or platelet lysate, and (ii) insulin or a substitute thereof, and transferrin or a substitute thereof, preferably insulin and transferrin. In particular, the liquid culture medium may further comprise

from 1% to 20% of plasma or serum, preferably from 2% to 10% of plasma or serum; or from 0.1% to 2% platelet lysate, preferably from 0.2% to 1% platelet lysate; and

from 5 μg/mL to 20 μg/mL of insulin, preferably from 8 μg/mL to 12 μg/mL; and

from 10 μg/mL to 100 μg/mL of transferrin, preferably from 30 μg/mL to 60 μg/mL of transferrin; and

The present invention also relates to the use of a liquid cell culture medium of the invention for the growth and/or differentiation of cells of the hematopoietic lineage, for the differentiation of an embryoid body, for the production of hematopoietic cell graft.

### BRIEF DESCRIPTION OF THE DRAWINGS

FIG. 1: Characterization of hiPSC-derived cells. (A) Experimental scheme. HiPSCs were differentiated into EBs over 17 days with the continuous presence of growth factors and cytokines. EB cells were characterized at different time points using q-PCR and flow cytometry. Images depicted representative EBs at D13 and 17 respectively. (B) Hierarchical clustering summarizing the expression of the set of 49 genes characteristics of the endothelium, hemogenic endothelium and hematopoietic cells with time in D3, D7, D9, D13, D15 and D17 EBs and in CD34⁺ cord blood cells. (C) Q-PCR patterns on genes representative of the EHT balance from D13 to 17 EB cell differentiation. For each gene, the fold change is the mean+/−SEM of 6 experiments. (D) Flow cytometry analysis of human CD309, ITGA2, MPL and CKIT at D13 and 17 of EB culture. (E) Flow cytometry analysis of the expression of APLNR and CXCR4 from D7 to day 17 of EB culture.

FIG. 2: Functional endothelio-hematopoietic profiling between D15 and D17. (A) In vitro tests probing the presence of endothelial (1-3) and hematopoietic (4-5) progenitors in EBs with time. Dissociated D15-17 EB cells generate (1) CFC-ECs, (2) pseudo-microtubules , (3) EC-like cells capable of several passages, (4) CFC, and (5) LTC-ICs. (B) Experimental scheme for in vivo tests to probe the endothelial capacity of D16 cells. (C) D16 cells/hMSCs plug section. Masson's trichrome staining (D) D16 cells/hMSCs plug section. Human von Willebrand factor⁺ cells (blue) immunostaining (E) D16 cells/hMSCs plug section Human CD31⁺ cells (red).

FIG. 3: In vivo engraftment of D17 EB cells in immunocompromised NSG mice. (A) Experimental design. (B) Representative flow cytometry analysis of human vs mouse CD45⁺ cell engraftment in a primary recipient. (C-D) Percentages of hCD34⁺ hCD43⁺ hCD45⁺ cells in primary (C) or secondary (D) mouse bone marrow 20 weeks post graft. Data are the mean+/−SEM. (E) Human hematopoietic lineage distribution in primary and secondary recipients. Numbers are normalized to 100%. (F) Clonogenic tests on BM cells isolated from primary and secondary recipients. Frequency of CFU-GM, BFU-E and CFU-GEMM colonies. (G) Representative colonies of CFU-GEMM (1), BFU-E (2) and CFU-GM (3) from primary and secondary BM recipients. (H) Cytospins. May Grünwald-Giemsa staining of cells isolated from clonogenic tests performed on primary and secondary recipient. Mature macrophages (1), histiomonocytes (2), myelocytes (2) and erythroblasts (3). (I) Human globin expression in CB CD34⁺ erythroid culture, BM from primary and secondary recipient and BFU-E from BM of primary recipients. Data are mean+/−SEM. (J) Maturation of human T cells. hCD2⁺ peripheral blood sorted cells stained with antibodies against hTCR αβ and hTCR γδ. (K) Functionality of human T cells. The whole thymus population is CFSE labeled at DO and gated on hCD3⁺ (green). At D5, the unstimulated population is red while the stimulated parent population is blue.

FIG. 4: Functional and molecular characterization of the APLNR⁺ population. (A) Correlation between the percentage of APLNR⁺ cells in the inoculum to those of hCD45⁺ cells in the NOD-SCID BM primary recipients, 18weeks post-graft. (B) Engraftment capacities of the APNLR⁺ (n=6, blue dots) and APNLR⁻ (n=4, red dots) populations. Cells containing the reconstitution potential are in the APLNR⁺ population. Data are expressed as the mean+/−SEM percentages of human engraftment, 18 weeks after transplant. (C) Combinatorial flow cytometry analysis of the APLNR⁺ population using CD45, TIE, ENG and CKIT anti-human antibodies. (D) PCA with the set of 49 mRNAs as variables and the six cell populations as observations. PC1 versus PC2 score plot. The PC1 dimension likely corresponds to the trait «hematopoietic differentiation» which accounts for 44.9% of the variance. HiPSCs are segregated from the main axis. (E) PCA with the set of 49 mRNAs as variables and the populations endowed or not with grafting potential. The PC3 dimension which accounts for 19.23% of the variance segregates the two groups. (F) Heat map of the 8 genes permitting the segregation of the two groups.

FIG. 5: Characterization of the APLNR⁺ and ⁻ populations. Representative profile of hCD45 and hCD43 expression in mice BM grafted with either the D17 APLNR⁻ (left) or ⁺ cell fractions.

FIG. 6: Unsupervised principal component analysis performed on 5859 differential genes between the groups allowed to significantly discriminated sample groups with a p-value of 4.75E-8 on the principal map.

FIG. 7: Circosplot describing supervised analysis by Significance analysis for microarray (SAM) between each xeno-transplant group and HSC group was performed in order found HSCs biomarkers in each group of SRCs-IPSCs.

FIG. 8: Venn diagram which compared HSCs biomarkers enriched in each group of SRC-IPSCs showed any gene in common

FIG. 9: Experimental design of In vivo engraftment of sorted D17 EB cells in immunocompromised NSG mice.

FIG. 10: Percentages of hCD34⁺ hCD43⁺ hCD45⁺ cells in primary or secondary mouse bone marrow 20 weeks post graft. Data are the mean+/−SEM.

### DETAILED DESCRIPTION OF THE INVENTION

The first transplantable HSCs are produced during embryonic development from a specialized population of endothelial cells (ECs) called the hemogenic endothelium. Following endothelial-to-hematopoietic transition (EHT), these hemogenic ECs differentiate into hematopoietic cells (HCs) including HSCs, enter the circulation, amplify in the fetal liver, and attain the BM, their definitive site of residence. These early steps of developmental hematopoiesis are fully recapitulated in embryoid body (EB) cultures, notably, the generation of hemogenic ECs and the budding of HCs.

The inventors herein developed a one step, vector-free and stromal-free system procedure to direct differentiation of human induced pluripotent stem cells (hiPSCs) into the endo-hematopoietic lineage. While in standard protocols CD34+CD45+ progenitors appeared from bursting EBs at day (D) 10 until day 14, the culture conditions applied by the inventors provided D17 embryoid bodies exhibited well defined compact spherical structure without burst therefore assessing a dramatic delay in the differentiation process. These culture conditions were applied to three different hiPSCs cell lines differing by their reprogramming protocols e.g. episomal or retroviral, with similar differentiation efficacies hence demonstrating the sturdiness of the method.

Based on the analysis of these differentiated embryoid body cells as well as bioinformatic analysis of the transcriptome of hematopoietic stem cells capable of only primary engraftment or primary and secondary engraftments, the inventors herein identified a sub-fraction of early primitive hematopoietic stem cells that displayed not only a high engraftment capacity but also a robust and prolonged self-renewal capacity, making these cells an ideal source for hematopoietic transplantation. They found that this sub-fraction can be characterized by the expression of Fms-Like Tyrosine kinase 3 receptor (FLT3 or CD135), and/or the thrombopoietin receptor (MPL or CD110) and/or the apelin receptor (APLNR).

Accordingly, in a first aspect, the present invention relates to a method, preferably an in vitro method, of preparing hematopoietic cell graft, comprising

a) providing a population of cells comprising hematopoietic stem cells, and

b) sorting cells of said population based on cell surface antigens CD135 and/or CD110, and/or the expression of the apelin receptor (APLNR), preferably based on cell surface antigen CD110, and

c) recovering cells that are CD135+ and/or CD110+ and/or APLNR+, preferably CD110+.

The present invention also relates to a method, preferably an in vitro method, of enriching a population of cells for hematopoietic stem cells that are suitable for hematopoietic transplantation, i.e. that are capable of long-term multilineage engraftment and self-renewal, comprising

a) providing a population of cells comprising hematopoietic stem cells, and

b) sorting cells of said population based on cell surface antigens CD135 and/or CD110, and/or the expression of the apelin receptor (APLNR), preferably based on cell surface antigen CD110, and

c) recovering cells that are CD135+, CD110+ and/or APLNR+, preferably CD110+.

CD135+ and/or CD110+ and/or APLNR+ recovered cells may be used as hematopoietic graft or may be included in or added to a hematopoietic graft (e.g. a bone marrow or cord blood transplant) in order to improve potency of said graft.

As used herein, the term “CD135” or “FLT3” refers to the class III receptor tyrosine kinase activated by binding of the cytokine Flt3 ligand (FLT3L) to the extracellular domain. In humans, this gene is encoded by the FLT3 gene (Gene ID: 2322). Upon activation, CD135 phosphorylates and activates multiple cytoplasmic effector molecules in pathways involved in apoptosis, proliferation, and differentiation of hematopoietic cells in bone marrow. Mutations that result in the constitutive activation of this receptor result in acute myeloid leukemia and acute lymphoblastic leukemia

As used herein, the term “CD110” or “MPL” refers to the thrombopoietin receptor also known as the myeloproliferative leukemia protein. In humans, CD110 is encoded by the MPL (myeloproliferative leukemia virus) oncogene (Gene ID: 4352). CD110 is a 635 amino acid transmembrane domain, with two extracellular cytokine receptor domains and two intracellular cytokine receptor box motifs. Its ligand, i.e. thrombopoietin, was shown to be the major regulator of megakaryocytopoiesis and platelet formation.

The term “APLNR”, as used herein, refers to the apelin receptor, i.e. a G protein-coupled receptor which binds apelin. This receptor was shown to be involved in the cardiovascular and central nervous systems, in glucose metabolism, in embryonic and tumor angiogenesis and as a human immunodeficiency virus coreceptor. In humans, this receptor is encoded by the APLNR gene (Gene ID: 187).

As used herein, the term “hematopoietic cell graft” or “hematopoietic graft” refers to an ex vivo cellular product to be used for hematopoietic transplantation. A hematopoietic cell graft may comprise hematopoietic stem cells obtained from mobilized peripheral blood, placental blood, umbilical cord blood, amniotic fluid, bone marrow, liver and/or spleen as well as immortalized HSC and/or HSC obtained from differentiation of pluripotent stem cells (e.g. induced pluripotent stem cells) and/or embryonic stem cells.

The population of cells provided in step a) comprises hematopoietic stem cells (HSC) and, in particular, early primitive HSC.

Preferably, the population of cells provided in step a) is a population of human cells.

As used herein, the term “hematopoietic stem cell” or “HSC” refers to a cell possessing the ability of both multipotency and self-renewal. Multi-potency is the ability to differentiate into all functional blood cells, e.g. B cells, T cells, NK cells, lymphoid dendritic cells, myeloid dendritic cells, granulocytes, macrophages, megakaryocytes and erythroid cells. Self-renewal is the ability to give rise to HSC itself without differentiation.

The term “early primitive HSC”, as used herein, refers to a HSC which is a precursor of CD34+/CD45+ HSCs and possesses the ability of both multipotency and self-renewal. An early primitive HSC belongs to the hemogenic endothelium capable of endothelial to hematopoietic transition and may be CD34−/CD45− or CD34+/CD45−. Early primitive HSC may also express CXCR4 and/or display up-regulation of genes involved in early hematopoietic commitment (e.g. HOXB4, c-MYC and MITF), self-renewal (e.g. HOXA9, ERG and RORA) and stemness (e.g. SOX4 and MYB) and/or may be a long-term culture-initiating cell (LTC-IC), i.e. a HSC which is able to generate colony-forming unit-cells (CFU) after 5 to 8 weeks (35 to 60 days) of culture on bone marrow (BM) stroma (Miller and Eaves, Methods Mol Med. 2002; 63:123-41). In some preferred embodiments, the term “early primitive HSC” refers to CD34−/CD45− or CD34+/CD45− LTC-IC cells. In some other embodiments, the term “early primitive HSC” may also refer to CD34+/CD45+ or CD34−/CD45+ LTC-IC cells.

The population of cells provided in step a) may comprise HSC obtained from peripheral blood, placental blood, umbilical cord blood, amniotic fluid, bone marrow, liver and/or spleen, immortalized HSC, pluripotent stem cells and/or embryonic stem cells.

In an embodiment, the population of cells provided in step a) comprises, or consists of, cells obtained from peripheral blood, placental blood, umbilical cord blood, amniotic fluid, bone marrow, liver and/or spleen, preferably cells obtained from peripheral blood, placental blood, umbilical cord blood and/or bone marrow. In particular, the population of cells provided in step a) may be a population of cells obtained from peripheral blood, placental blood, umbilical cord blood, amniotic fluid, bone marrow, liver or spleen, preferably a population of cells obtained from peripheral blood, placental blood, umbilical cord blood or bone marrow.

HSC may be obtained from the different sources hereabove mentioned using any method known by the skilled person.

For example, peripheral blood stem cells may be found in total blood sample or may be collected from the blood through a process known as apheresis. The peripheral stem cell yield may be boosted with administration of a compound stimulating the migration of stem cells from the donor's bone marrow into the peripheral circulation. Such compounds include for example granulocyte-colony stimulating factor or Mozobil™ (Plerixafor). After such treatment, peripheral blood is usually named “mobilized peripheral blood”.

HSC may also be obtained from bone marrow of a subject. In this case, the HSC are removed from a large bone of the subject, typically the pelvis, through a large needle that reaches the center of the bone.

Umbilical cord blood or placental blood may be obtained when a mother donates her infant's umbilical cord and placenta after birth. Cord or placental blood has a higher concentration of HSC than is normally found in adult blood.

In a more particular embodiment, the population of cells provided in step a) is a sample of peripheral blood, preferably mobilized peripheral blood, bone marrow, umbilical cord blood or placental blood.

In another embodiment, the population of cells provided in step a) comprises, or consists of, immortalized HSC, preferably human immortalized HSC. HSC may be immortalized using any method known by the skilled person such as retroviral-mediated gene transfer of beta-catenin (Templin et al. Exp Hematol. 2008 February; 36(2):204-15).

In a further embodiment, the population of cells provided in step a) comprises, or consists of, HSC obtained from in vitro differentiation of pluripotent stem cells, preferably selected from induced pluripotent stem cells or embryonic stem cells, more preferably induced pluripotent stem cells.

Producing HSC from human embryonic stem cells may meet ethical challenges. In an embodiment, embryonic stem cells are non-human embryonic stem cells. In another embodiment, embryonic stem cells are human embryonic stem cells with the proviso that the method itself or any related acts do not include destruction of human embryos.

Embryonic stem cells are derived from the inner cell mass of the pre-implantation blastocyst. Embryonic stem cells are able to maintain an undifferentiated state or can be directed to mature along lineages deriving from all three germ layers, ectoderm, endoderm and mesoderm. hESCs possess indefinite proliferative capacity in vitro, and have been shown to differentiate into the hematopoietic cell fate, giving rise to erythroid, myeloid, and lymphoid lineages using a variety of differentiation procedures (Bhatia, Hematology Am Soc Hematol Educ Program. 2007:11-6).

In a preferred embodiment, the population of cells provided in step a) comprises, or consists of, HSC obtained from differentiation of induced pluripotent stem cells (iPSC), preferably from differentiation of human iPSC.

iPSC are derived from a non-pluripotent cell, typically an adult somatic cell, by a process known as reprogramming, where the introduction of only a few specific genes are necessary to render the cells pluripotent. Various combinations of genes were shown to render the cells pluripotent such as Oct4/Sox2/Nanog/Lin28 or Oct4/Sox2/KLF/cMyc. One benefit of use of iPSC is the avoidance of the use of embryonic cells altogether and hence any ethical questions thereof.

iPSC may be obtained from the subject to be treated (transplant patient) or from another subject. Preferably, iPSC are derived from cells from the subject to be treated, in particular from fibroblasts of this subject.

Pluripotent stem cells, and in particular iPSC or embryonic stem cells, may be differentiated into HSC, or more particularly into early primitive HSC, using any method known by the skilled person such as any method described in Bathia (supra), Doulatov et al. (Cell Stem Cell. 2013 Oct. 3; 13(4): 10.1016) or Sandler et al. (Nature. 2014 Jul. 17; 511(7509):312-8).

In a particular embodiment, the method of the invention further comprises, before step a),

providing pluripotent stem cells, in particular iPSC or embryonic stem cells, preferably human iPSC or human embryonic stem cells, more preferably human iPSC,

inducing embryoid body (EBs) formation,

culturing EBs in a liquid culture medium triggering differentiation of the pluripotent stem cells into the endo-hematopoietic lineage, and

dissociating EB cells,

thereby obtaining a population of cells provided in step a) of the method of the invention and as described above.

The formation of embryoid bodies from pluripotent stem cells may be obtained by any protocol known by the skilled person. For example, pluripotent stem cells may be treated with collagenase IV and transferred to low attachment plates in liquid culture medium.

Differentiation of the pluripotent stem cells into the endo-hematopoietic lineage is then obtained by culturing embryoid bodies in a liquid culture medium triggering said differentiation. This liquid culture medium may be the same as the culture medium used during the formation of embryoid bodies.

Several culture media triggering the differentiation of the pluripotent stem cells into the endo-hematopoietic lineage have been described (see e.g. Lapillonne et al., haematological, 2010; 95(10), Doulatov et al., Cell Stem Cell. 2013, 13(4)) and can be used in the present invention.

However, the inventors found that culture medium comprising a specific combination of cytokines and growth factors provides differentiated embryoid bodies exhibited well-defined compact spherical structure without burst. Thus, in a particular embodiment, the culture medium triggering the differentiation of the pluripotent stem cells into the endo-hematopoietic lineage, comprises stem cell factor (SCF), thrombopoietin (TPO), FMS-like tyrosine kinase 3 (FLT3) ligand, bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1). This culture medium may further comprise plasma, serum, platelet lysate, serum albumin, transferrin or a substitute thereof and/or insulin or a substitute thereof, preferably (i) plasma, serum and/or platelet lysate, and (ii) transferrin and insulin.

In a preferred embodiment, the culture medium triggering the differentiation of the pluripotent stem cells into the endo-hematopoietic lineage is the culture medium of the present invention and described hereafter.

Preferably, embryoid bodies are cultured in the liquid culture medium for 14 to 19 days, more preferably for 15 to 18 days, and even more preferably for 17 days. In a preferred embodiment, embryoid bodies are cultured in the liquid culture medium of the invention and described hereafter for 14 to 19 days, more preferably for 15 to 18 days, and even more preferably for 17 days.

Differentiated embryoid bodies are then dissociated, for example by incubation with collagenase B and cell dissociation buffer, or any other method known by the skilled person.

As demonstrated in the experimental section of the present application, the population of dissociated cells comprises HSC, and in particular early primitive HSC, and may be provided in step a) of the method of the present invention.

The presence of early primitive HSC in a population of cells comprising HSC may be assessed by any method known by the person skilled in the art, for example using the long-term culture initiating cell (LTC-IC) assay as described in Liu et al. Methods Mol Biol. 2013; 946:241-56.

HSC, including early primitive HSC, may be stored before to be used in the method of the invention. In particular, cells can be cryopreserved for prolonged periods, optionally in the presence of a cryo-preservative such as DMSO.

In step b) of the method of invention, cells of the population provided in step a) and as described above, are sorted based on the expression of cell surface antigens CD135 and/or CD110, and/or the expression of APLNR.

As used herein, the term “sorting” of cells, refers to an operation that segregates cells into groups according to a specified criterion such as marker expression. Any method known by the skilled person to segregate cells according to the specified criterion may be used, including but not limited to, fluorescent activated cell sorting (FACS) or magnetic-activated cell sorting (MACS). As used herein, the expression “sorting based on the expression of” a particular protein, e.g. a cell surface antigen, refers to an operation that segregates cells expressing said protein and cells that do not express said protein. In preferred embodiments, expression of CD135, CD110 or APLNR is detected at the surface of the cell. However, any other method known by the skilled person and allowing detection of this expression may be used such as methods detecting specific mRNA (e.g. RT-PCR).

Cells may be sorted based on

the expression of cell surface antigens CD135 and CD110, and optionally the expression of APLNR; or

the expression of cell surface antigen CD135, and optionally the expression of APLNR and CD110; or

the expression of cell surface antigen CD135, and optionally the expression of APLNR; or

the expression of cell surface antigen CD135, and optionally the expression of CD110; or

the expression of cell surface antigen CD110, and optionally the expression of APLNR; or

the expression of cell surface antigen CD110, and optionally the expression of cell surface antigen CD135; or

the expression of cell surface antigen CD110, and optionally the expression of APLNR and CD135; or

the expression of cell surface antigens CD135 and CD110, and the expression of APLNR; or

the expression of cell surface antigen CD135 and the expression of APLNR; or

the expression of cell surface antigen CD110 and the expression of APLNR; or

the expression of APLNR, and optionally the expression of cell surface antigens CD135 and CD110; or

the expression of APLNR, and optionally the expression of cell surface antigen CD135; or

the expression of APLNR, and optionally the expression of cell surface antigen CD110.

In embodiments wherein cells are sorted based on the expression of two or three markers, e.g. CD135, CD110 and APLNR, the selection based on each of these markers may be simultaneous or sequential, in any order.

In a particular embodiment, in step b) cells are sorted based on the expression of cell surface antigens CD135 and/or CD110, preferably CD135 or CD110. In a preferred embodiment, in step b) cells are sorted based on the expression of cell surface antigen CD110, and optionally on the expression of cell surface antigen CD135. In these embodiments, the method may further comprise before, after or simultaneously to step b), sorting cells based on the expression of APLNR.

In step c) of the method of the invention, cells which are CD135+ and/or CD110+ and/or APLNR+ are recovered.

In a preferred embodiment, cells which are CD110+ are recovered.

As used herein, the term “+” refers to the expression of the marker of interest, preferably at the cell surface. For example, CD135+ cells are cells that express the cell surface antigen CD135, and CD110+/APLNR+ cells are cells that express the cell surface antigen CD110 and APLNR. On the contrary, the term “−” refers to the lack of expression of the marker of interest, preferably at the cell surface. For example, CD135− cells are cells that do not express the cell surface antigen CD135, and CD110+/APLNR− cells are cells that express the cell surface antigen CD110 and do not express APLNR.

According to the method used to sort cells, steps b) and c) may be sequential or simultaneous.

According to the marker used during the sorting step, recovered cells may be CD135+ cells, CD110+ cells, APLNR+ cells, CD135+/CD110+ cells, CD135+/APLNR+cells, CD110+/APLNR+ cells, or CD135+/CD110+/APLNR+ cells. Preferably, cells are CD110+ cells, CD135+/CD110+ cells, CD110+/APLNR+ cells or CD135+/CD110+/APLNR+ cells.

These cells are capable of long-term multilineage engraftment and self-renewal and may be used for HSC transplantation.

If necessary, the method of the invention may comprise several successive sorting steps based on the expression of CD135, CD110 and/or APLNR in order to enrich the cellular product for CD135+, CD110+ and/or APLNR+ HSC.

Optionally, before use, these cells may be stored, in particular may be cryopreserved for short or prolonged periods, optionally in the presence of a cryo-preservative such as DMSO.

In another aspect, the present invention also relates to a method, preferably an in vitro method, of identifying and/or selecting hematopoietic stem cells that are suitable for hematopoietic transplantation, i.e. that are capable of long-term multilineage engraftment and self-renewal, comprising

a) providing a population of cells comprising hematopoietic stem cells, and

b) assessing said cells for the expression of cell surface antigens CD135 and/or CD110, and/or the expression of the apelin receptor (APLNR), preferably for the expression of cell surface antigen CD110, and

c) identifying and/or selecting cells that are CD135+ and/or CD110+ and/or APLNR+, preferably CD110+.

All embodiments described above for the method of preparing a hematopoietic cell graft of the invention are also encompassed in this aspect.

Expression of cell surface antigens CD135 and/or CD110, and/or the expression of the apelin receptor (APLNR) may be assessed by any method known by the skilled person such as FACS, MACS, immunohistochemistry, Western-blot, protein or antibody arrays, RT-PCR or by transcriptome approaches.

According to the method used to assess expression of CD135, CD110 or APLNR, steps b) and c) may be sequential or simultaneous. For example, using FACS or MACS, detection of the expression and selection may be simultaneous.

CD135+ and/or CD110+ and/or APLNR+ identified and/or selected cells may be used as hematopoietic graft or may be included in or added to a hematopoietic graft (e.g. a bone marrow or cord blood transplant) in order to improve potency of said graft.

In a further aspect, the present invention also relates to a method, preferably an in vitro method, of producing transplantable HSC from pluripotent stem cells comprising

providing pluripotent stem cells, in particular iPSC or embryonic stem cells, preferably human iPSC or human embryonic stem cells, more preferably human iPSC,

inducing embryoid body (EBs) formation,

culturing EBs in a liquid culture medium triggering differentiation of the pluripotent stem cells into the endo-hematopoietic lineage,

dissociating EB cells,

sorting dissociated EB cells based on cell surface antigens CD135 and/or CD110, and/or the expression of the apelin receptor (APLNR), preferably based on cell surface antigen CD110, and

recovering cells that are CD135+, CD110+ and/or APLNR+, preferably CD110+.

All embodiments described above for the method of preparing a hematopoietic cell graft of the invention are also encompassed in this aspect.

As used herein, the term “transplantable HSC” refers to hematopoietic stem cells that are suitable for hematopoietic transplantation, i.e. that are capable of long-term multilineage engraftment and self-renewal.

Preferably, the culture medium triggering the differentiation of the pluripotent stem cells into the endo-hematopoietic lineage, comprises stem cell factor (SCF), thrombopoietin (TPO), FMS-like tyrosine kinase 3 (FLT3) ligand, bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1). This culture medium may further comprise plasma, serum, platelet lysate, serum albumin, transferrin or a substitute thereof and/or insulin or a substitute thereof, preferably (i) plasma, serum and/or platelet lysate, and (ii) transferrin and insulin.

In a preferred embodiment, the culture medium triggering the differentiation of the pluripotent stem cells into the endo-hematopoietic lineage is the culture medium of the present invention and described hereafter.

Preferably, embryoid bodies are cultured in the liquid culture medium for 14 to 19 days, more preferably for 15 to 18 days, and even more preferably for 17 days. In a preferred embodiment, embryoid bodies are cultured in the liquid culture medium of the invention and described hereafter for 14 to 19 days, more preferably for 15 to 18 days, and even more preferably for 17 days.

As demonstrated herein, CD135+, CD110+ and/or APLNR+ HSC are long-term multipotent HSC supporting multi-lineage hematopoietic reconstitution and self-renewal in vivo, and thus constitute an excellent source of cells for HSC transplantation.

Thus, in another aspect, the present invention relates to the use of CD135, CD110 and/or APLNR as markers of hematopoietic stem cells that are suitable for hematopoietic transplantation, i.e. that are capable of engraftment, and in particular of long-term multilineage engraftment and self-renewal.

The invention also relates to the use of CD135, CD110 and/or APLNR as markers to assess the potency of a hematopoietic cell graft and/or as markers to predict hematopoietic graft outcome and/or performance.

The present invention further relates to a method, preferably an in vitro method, of assessing the potency of a hematopoietic cell graft, comprising assessing a hematopoietic cell graft for the presence or the absence of HSC expressing CD135, CD110 and/or APLNR, preferably for the presence or the absence of HSC expressing CD110, i.e. the presence or the absence of cells capable of long-term multilineage engraftment and self-renewal, the absence of said cells being indicative of low or lack of potency. Inversely, the presence of HSC expressing CD135, CD110 and/or APLNR can be considered as indicative of a good potency.

As used herein, the term “potency” refers to the specific capacity of a cellular product to affect a given result, and in particular to the capacity of a hematopoietic cellular product to provide, upon transplantation, in vivo multi-lineage hematopoietic reconstitution and self-renewal, i.e. to regenerate the immune-hematopoietic system in the transplant patient.

Transplantation of a hematopoietic cell graft of low or lack of potency may result in graft failure. As a consequence, a hematopoietic cell graft which does not comprise any HSC expressing CD135, CD110 and/or APLNR should not be used in transplantation.

The present invention also relates to a method, preferably an in vitro method, of predicting the outcome of HSC transplantation, comprising detecting in a hematopoietic cell graft the presence or the absence of HSC expressing CD135, CD110 and/or APLNR, preferably expressing CD110, the absence of said cells being indicative of a poor prognosis, i.e. high risk of graft failure. Inversely, the presence of HSC expressing CD135, CD110 and/or APLNR can be considered as indicative of a good prognosis.

As used herein, the term “poor prognosis” refers to a decreased patient survival and/or high risk of graft failure, i.e. to a high risk that the transplantation fails to regenerate the immune-hematopoietic system in the transplant patient. Inversely, the term “good prognosis” refers to an increased patient survival and an increased probability of success of transplantation, i.e. an increased probability that the transplantation allows regeneration of the immune-hematopoietic system in the transplant patient.

The present invention further relates to a method, preferably an in vitro method, of predicting engraftment potential of a hematopoietic cell graft comprising detecting in a hematopoietic cell graft the presence or the absence of HSC expressing CD135, CD110 and/or APLNR, preferably expressing CD110, the absence of said cells being indicative of a poor engraftment potential, i.e. high risk of graft failure. Inversely, the presence of HSC expressing CD135, CD110 and/or APLNR can be considered as indicative of a good engraftment potential, i.e. an increased probability of success of transplantation.

The presence or absence of HSC expressing CD135, CD110 and/or APLNR may be assessed by any method known by the skilled person or described above. For example, CD135+, CD110+ and/or APLNR+ cells may be detected using fluorescent activated cell sorting (FACS), magnetic-activated cell sorting (MACS), or any immunoassay using antibodies directed against CD135, CD110 or APLNR. Monoclonal antibodies directed against CD135, CD110 or APLNR are commercially available.

The methods of assessing the potency of a hematopoietic cell graft, predicting the outcome of HSC transplantation, or predicting engraftment potential of a hematopoietic cell graft as described above may further comprise any other phenotyping or functional assays routinely used by the skilled person such as counting the total number of viable nucleated cells (TNC), and/or measuring the total number of viable CD34+ cells, and/or measuring the number of functional progenitor cells able to produce colonies of hematopoietic cells in methylcellulose-based culture medium supplemented with stimulatory growth factors (CFU assay), and/or measuring the LTC-IC frequency.

In another aspect, the present invention relates to a hematopoietic cell graft prepared according to any method of the invention.

It further relates to a hematopoietic cell graft wherein at least 1, 5, 10, 20, 30, 40, 50, 60 or 70% of cells are CD135+, CD110+ and/or APLNR+ hematopoietic stem cells, preferably CD110+ hematopoietic stem cells, and a pharmaceutically acceptable carrier. Preferably, at least 70%, 75%, 80%, 85%, 90%, 95% or 99% of cells of the hematopoietic cell graft are CD135+, CD110+ and/or APLNR+ hematopoietic stem cells, preferably

CD110+ hematopoietic stem cells. More preferably, at least 70% 75%, 80%, 85%, 90%, 95% or 99% of cells of the hematopoietic cell graft are CD135+ and/or CD110+ HSC, preferably CD110+ HSC.

The proportion of CD135+, CD110+ and/or APLNR+ HSC may be easily determined using any method known by the skilled person or described herein.

As used herein, the term “pharmaceutically acceptable” means approved by a regulatory agency or recognized pharmacopeia such as European Pharmacopeia, for use in animals and/or humans. The term “carrier” or “excipient” refers to a diluent, adjuvant, carrier, or vehicle with which the cells are administered. As is well known in the art, pharmaceutically acceptable excipients are relatively inert substances, preferably injectable substances well known by the skilled person.

All embodiments described above for the method of preparing a hematopoietic cell graft of the invention are also encompassed in this aspect.

In a further aspect, the present invention relates to a hematopoietic cell graft of the invention for use in the treatment of various disorders related to deficiencies in hematopoiesis caused by disease, condition, or myeloablative treatments, in particular for the treatment of malignant or non-malignant diseases.

It also relates to the use of a hematopoietic cell graft of the invention, for preparing a medicament for treating disorders related to deficiencies in hematopoiesis caused by disease, condition, or myeloablative treatments, in particular for treating malignant or non-malignant diseases.

It further relates to a method for treating disorders related to deficiencies in hematopoiesis caused by disease, condition, or myeloablative treatments, in particular for treating malignant or non-malignant diseases, in particular for treating a malignant or non-malignant disease, in a subject in need thereof, comprising administering an effective amount of hematopoietic cell graft of the invention, to the subject.

It also relates to a method for treating disorders related to deficiencies in hematopoiesis caused by disease, condition, or myeloablative treatments, in particular for treating malignant or non-malignant diseases, in a subject in need thereof, comprising assessing the potency of a hematopoietic cell graft according to the method of the invention described above and, if the potency is good, administering an effective amount of said hematopoietic cell graft to the subject.

All embodiments described above for the method of preparing a hematopoietic cell graft of the invention, for assessing the potency of a hematopoietic cell graft or for the hematopoietic cell graft of the invention are also encompassed in this aspect.

Examples of malignant diseases include, but are not limited to, multiple myeloma, non-Hodgkin's lymphoma, Hodgkin's disease, acute myeloid leukemia, acute lymphoblastic leukemia, chronic myeloid leukemia, myelodysplastic syndromes, myeloproliferative disorders, chronic lymphocytic leukemia, juvenile chronic myeloid leukemia, neuroblastoma, ovarian cancer and germ-cell tumors.

Examples of non-malignant diseases include, but are not limited to, autoimmune disorders, amyloidosis, aplastic anemia, paroxysmal nocturnal hemoglobinuria, Fanconi's anemia, Blackfan-Diamond anemia, thalassemia major, sickle cell anemia, severe combined immunodeficiency, Wiskott-Aldrich syndrome and inborn errors of metabolism.

The term “subject” or “patient” refers to an animal, preferably to a mammal, even more preferably to a human, including adult, child and human at the prenatal stage.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease. In some embodiment, this term may refer to the regeneration of the immune-hematopoietic system in the transplant patient.

By a “therapeutically efficient amount” is intended an amount of hematopoietic cell graft administered to a subject that is sufficient to constitute a treatment of the malignant or non-malignant disease as defined above. In some embodiments, this term may refer to the amount of hematopoietic cell graft necessary to regenerate the immune-hematopoietic system in the transplant patient.

The therapeutically efficient amount may vary according to the proportion of CD135+, CD110+ and/or APLNR+ cells in the hematopoietic cell graft, according to physiological data of the patient (e.g. age, size, and weight) and the disease to be treated.

In an embodiment, from 10⁴ to 10⁷, preferably from 10⁵ to 10⁷, and more preferably from 3.10⁵ to 6.10⁶, CD135+, CD110+ and/or APLNR+ cells/kg of body weight of the patient are administered. In a particular embodiment, from 10⁵ to 10⁶, preferably from 1.10⁵ to 5.10⁵, CD135+ and/or CD110+ cells , preferably CD110+ cells, per kg of body weight of the patient are administered. In another particular embodiment, from 10⁶ to 10⁷, preferably from 3.10⁶ to 8.10⁶, APLNR+ cells/kg of body weight of the patient are administered.

The hematopoietic cell graft according to the invention may be used in combination with other therapy such as other chemotherapy, immunotherapy, radiotherapy, or surgery, according to the disease to be treated.

The term “immunotherapy” refers to a therapeutic treatment stimulating the patient's immune system to attack the malignant tumor cells or cells responsible for the disease. It includes immunization of the patient with specific antigens (e.g. by administering a cancer vaccine), administration of molecules stimulating the immune system such as cytokines, or administration of therapeutic antibodies as drugs.

The term “radiotherapy” is a term commonly used in the art to refer to multiple types of radiation therapy including internal and external radiation therapy, radioimmunotherapy, and the use of various types of radiation including X-rays, gamma rays, alpha particles, beta particles, photons, electrons, neutrons, radioisotopes, and other forms of ionizing radiation. In particular, radiation therapy can be used to treat disease that may have spread outside the bone marrow, to relieve bone pain or for total body irradiation before a stem cell transplant.

The chemotherapy may be used to treat malignant disease and may include for example vincristine, daunorubicin, doxorubicin, idarubicin, mitoxantrone, cytarabine, asparaginase, etoposide, teniposide, mercaptopurine, methotrexate, cyclophosphamide, prednisone, dexamethasone, busalfan, hydroxyurea or interferon alpha, or any other relevant chemotherapy.

The hematopoietic cell graft may be used in autologous, syngeneic or allogeneic transplantation. As used herein, “allogeneic transplantation” refers to transplantation of cells deriving from or originating in a donor who is genetically non-identical to the recipient but of the same species. “Autologous transplantation” refers to transplantation of cells deriving from or originating in the same subject. The donor and the recipient is the same person. “Syngeneic transplantation” refers to transplantation of cells deriving from or originating in a donor who is genetically identical to the recipient.

In a particular embodiment, the hematopoietic cell graft is intended to be used in autologous transplantation and HSC, in particular CD135+, CD110 and/or APLNR+ cells, are derived from induced pluripotent stem cells originated from the subject to be treated.

In another particular embodiment, the hematopoietic cell graft is intended to be used in allogeneic transplantation and HSC, in particular CD135+, CD110 and/or APLNR+cells, are derived from placental or umbilical cord blood.

As shown in the experimental section, the inventors developed a liquid cell culture medium in which the differentiation process of embryoid bodies obtained from pluripotent stem cells, into the endo-hematopoietic lineage is delayed. This culture medium thus allows production and selection of early primitive hematopoietic stem cells that are capable of long-term multilineage engraftment and self-renewal in vivo, i.e. CD135+, CD110+ and/or APLNR+ HSC, under GMP-grade culture conditions.

Accordingly, in another aspect, the present invention also relates to a liquid cell culture medium comprising, or consisting essentially of, (i) plasma, serum, platelet lysate and/or serum albumin, and (ii) transferrin or a substitute thereof, insulin or a substitute thereof, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1). Preferably, the liquid cell culture medium comprises, or consists essentially of, (i) plasma, serum and/or platelet lysate, and (ii) transferrin, insulin, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

As used herein, the term “cell culture medium” relates to any culture medium, in particular any liquid culture medium, comprising a base culture medium liable to sustain the growth of eukaryotic cells, in particular mammalian cells, more particularly human cells. Base culture media are well known to one of skill in the art.

As used herein, the term “consisting essentially of” refers to a culture medium which comprises (i) plasma, serum, platelet lysate and/or serum albumin, preferably plasma, serum and/or platelet lysate, and (ii) transferrin or substitute thereof, preferably transferrin, insulin or substitute thereof, preferably insulin, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1), and does not comprise any other cytokines or growth factor.

In a particular embodiment, the culture medium of the invention comprises Iscove's Modified Dulbecco's Medium (IMDM) optionally complemented with glutamine or a glutamine-containing peptide, as base culture medium to which is added (i) plasma, serum, platelet lysate and/or serum albumin, preferably plasma, serum and/or platelet lysate, and (ii) transferrin or substitute thereof, preferably transferrin, insulin or substitute thereof, preferably insulin, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

Preferably, the culture medium of the invention comprises from 5 μg/mL to 20 μg/mL of insulin, more preferably from 8 μg/mL to 12 μg/mL, and even more preferably about 10 μg/mL of insulin. In preferred embodiments, insulin is human insulin, preferably human recombinant insulin.

Insulin substitute can be any compound known by the skilled person to fulfill the same function as insulin in a cellular culture medium. In particular, this substitute can be any insulin receptor agonist such as small molecule or aptamer agonists. Small molecule insulin receptor agonists have been described for example in Qiang et al. Diabetes. 2014 April; 63(4):1394-409, and aptamer agonists have been described for example in Yunn et al. Nucleic Acids Res. 2015 Sep. 18; 43(16):7688-701. Preferably, insulin is substituted by a zinc salt as described in Wong et al. Cytotechnology. 2004 July; 45(3):107-15. Examples of zinc salts include, but are not limited to, zinc chloride, zinc nitrate, zinc bromide or zinc sulfate. In preferred embodiment, insulin substitute is a zinc salt. The concentration of the insulin substitute depends on the nature of said compound and can be easily determined by the skilled person.

Preferably, the culture medium of the invention comprises from 10 μg/mL to 100 μg/mL of transferrin, preferably from 30 μg/mL to 60 μg/mL of transferrin, and even more preferably about 45 μg/mL of transferrin. In preferred embodiments, transferrin is iron-saturated human transferrin, preferably recombinant iron-saturated human transferrin.

Transferrin substitute can be any compound known by the skilled person to fulfill the same function as transferrin in a cellular culture medium. In particular, transferrin may be replaced by an iron chelator or an inorganic iron salt such as ferric citrate, ferric nitrate or ferrous sulfate. Examples of suitable iron chelators include, but are not limited to, ethylenediaminetetraacetic acid (EDTA), egtazic acid (EGTA), deferoxamine mesylate, dimercaprol or pentetic acid (DPTA). The concentration of the transferrin substitute depends on the nature of said compound and can be easily determined by the skilled person.

The culture medium may comprise plasma, serum, platelet lysate and/or serum albumin, preferably plasma, serum and/or platelet lysate, more preferably plasma or serum or platelet lysate or serum albumin, and even more preferably plasma or serum or platelet lysate. The culture medium may comprise from 1% to 20% of plasma or serum, preferably from 2% to 10% of plasma or serum, and more preferably about 5% of plasma or serum. In preferred embodiments, plasma or serum is human plasma or serum. Alternatively, or in addition, the culture medium may comprise from 0.1% to 2% platelet lysate, preferably from 0.2% to 1% platelet lysate, and more preferably about 0.5% platelet lysate. In preferred embodiments, platelet lysate is human platelet lysate. Alternatively, or in addition, the culture medium may comprise from 0.1% to 2% serum albumin, preferably from 0.5% to 1% serum albumin. In preferred embodiments, serum albumin is human serum albumin.

Preferably the culture medium of the invention comprises from 10 ng/mL to 100 ng/mL of SCF, more preferably from 10 ng/mL to 50 ng/mL of SCF, and even more preferably about 24 ng/mL of SCF. In preferred embodiments, SCF is human SCF, preferably recombinant human SCF.

Preferably the culture medium of the invention comprises from 10 ng/mL to 100 ng/mL of TPO, more preferably from 10 ng/mL to 50 ng/mL of TPO, and even more preferably about 21 ng/mL of TPO. In preferred embodiments, TPO is human TPO, preferably recombinant human TPO.

Preferably the culture medium of the invention comprises from 10 ng/mL to 100 ng/mL of FLT3-L, more preferably from 10 ng/mL to 50 ng/mL of FLT3-L, and even more preferably about 21 ng/mL of FLT3-L. In preferred embodiments, FLT3-L is human FLT3-L, preferably recombinant human FLT3-L.

Preferably the culture medium of the invention comprises from 50 ng/mL to 300 ng/mL of BMP4, more preferably from 150 ng/mL to 250 ng/mL of BMP4, and even more preferably about 194 ng/mL of BMP4. In preferred embodiments, BMP4 is human BMP4, preferably recombinant human BMP4.

Preferably the culture medium of the invention comprises from 50 ng/mL to 300 ng/mL of VEGF, more preferably from 150 ng/mL to 250 ng/mL of VEGF, and even more preferably about 200 ng/mL of VEGF. In preferred embodiments, VEGF is human VEGF, preferably recombinant human VEGF, and more preferably recombinant human VEGF-A165.

Preferably the culture medium of the invention comprises from 10 ng/mL to 100 ng/mL of IL3, more preferably from 20 ng/mL to 80 ng/mL of IL3, and even more preferably about 50 ng/mL of IL3. In preferred embodiments, IL3 is human IL3, preferably recombinant human IL3.

Preferably the culture medium of the invention comprises from 10 ng/mL to 100 ng/mL of IL6, more preferably from 20 ng/mL to 80 ng/mL of IL6, and even more preferably about 50 ng/mL of IL6. In preferred embodiments, IL6 is human IL6, preferably recombinant human IL6.

Preferably the culture medium of the invention comprises from 1 ng/mL to 20 ng/mL of IL1, more preferably from 1 ng/mL to 10 ng/mL of IL1, and even more preferably about 5 ng/mL of IL1. In preferred embodiments, IL1 is human IL1, preferably recombinant human IL1.

Preferably the culture medium of the invention comprises from 10 ng/mL to 200 ng/mL of GCSF, more preferably from 50 ng/mL to 150 ng/mL of GCSF, and even more preferably about 100 ng/mL of GCSF. In preferred embodiments, GCSF is human GCSF, preferably recombinant human GCSF.

Preferably the culture medium of the invention comprises from 1 ng/mL to 10 ng/mL of IGF1, more preferably from 1 ng/mL to 10 ng/mL of IGF1, and even more preferably about 5 ng/mL of IGF1. In preferred embodiments, IGF1 is human IGF1, preferably recombinant human IGF1.

In a particular embodiment, the liquid cell culture medium of the invention comprises

from 1% to 20% of plasma or serum, preferably from 2% to 10% of plasma or serum; or from 0.1% to 2% platelet lysate, preferably from 0.2% to 1% platelet lysate; or from 0.1% to 2% serum albumin, preferably from 0.5% to 1% serum albumin; and/or

from 5 μg/mL to 20 μg/mL of insulin or a substitute thereof, preferably insulin, preferably from 8 μg/mL to 12 μg/mL of insulin or a substitute thereof, preferably insulin; and/or

from 10 μg/mL to 100 μg/mL of transferrin or a substitute thereof, preferably transferrin, preferably from 30 μg/mL to 60 μg/mL of transferrin or a substitute thereof, preferably transferrin; and/or

from 10 ng/mL to 100 ng/mL of SCF, preferably from 10 ng/mL to 50 ng/mL of SCF; and/or

from 10 ng/mL to 100 ng/mL of TPO, preferably from 10 ng/mL to 50 ng/mL of TPO; and/or

from 10 ng/mL to 100 ng/mL of FLT3-L, preferably from 10 ng/mL to 50 ng/mL of FLT3-L; and/or

from 100 ng/mL to 500 ng/mL of BMP4, preferably from 150 ng/mL to 250 ng/mL of BMP4; and/or

from 50 ng/mL to 300 ng/mL of VEGF, preferably from 150 ng/mL to 250 ng/mL of VEGF; and/or

from 10 ng/mL to 100 ng/mL of IL3, preferably from 20 ng/mL to 80 ng/mL of IL3; and/or

from 10 ng/mL to 100 ng/mL of IL6, preferably from 20 ng/mL to 80 ng/mL of IL6; and/or

from 1 ng/mL to 20 ng/mL of IL1, preferably from 1 ng/mL to 10 ng/mL of IL1; and/or

from 10 ng/mL to 200 ng/mL of GCSF, preferably from 50 ng/mL to 150 ng/mL of GCSF; and/or

from 1 ng/mL to 20 ng/mL of IGF1, preferably from 1 ng/mL to 10 ng/mL of IGF1.

Preferably, the medium meets all these features.

In another particular embodiment, the liquid cell culture medium of the invention comprises

from 1% to 20% of plasma or serum, preferably from 2% to 10% of plasma or serum; or from 0.1% to 2% platelet lysate, preferably from 0.2% to 1% platelet lysate; and/or

from 5 μg/mL to 20 μg/mL of insulin, preferably from 8 μg/mL to 12 μg/mL; and

from 10 μg/mL to 100 μg/mL of transferrin, preferably from 30 μg/mL to 60 μg/mL of transferrin; and/or

from 10 ng/mL to 100 ng/mL of SCF, preferably from 10 ng/mL to 50 ng/mL of SCF; and/or

from 10 ng/mL to 100 ng/mL of TPO, preferably from 10 ng/mL to 50 ng/mL of TPO; and/or

from 100 ng/mL to 500 ng/mL of FLT3-L, preferably from 250 ng/mL to 350 ng/mL of FLT3-L; and/or

from 10 ng/mL to 100 ng/mL of BMP4, preferably from 10 ng/mL to 50 ng/mL of BMP4; and/or

from 50 ng/mL to 300 ng/mL of VEGF, preferably from 150 ng/mL to 250 ng/mL of VEGF; and/or

from 10 ng/mL to 100 ng/mL of IL3, preferably from 20 ng/mL to 80 ng/mL of IL3; and/or

from 10 ng/mL to 100 ng/mL of IL6, preferably from 20 ng/mL to 80 ng/mL of IL6; and/or

from 1 ng/mL to 20 ng/mL of IL1, preferably from 1 ng/mL to 10 ng/mL of IL1; and/or

from 10 ng/mL to 200 ng/mL of GCSF, preferably from 50 ng/mL to 150 ng/mL of GCSF; and/or

from 10 ng/mL to 150 ng/mL of IGF1, preferably from 10 ng/mL to 100 ng/mL of IGF1.

Preferably, the medium meets all these features.

In another particular embodiment, the liquid cell culture medium of the invention comprises

from 1% to 20% of plasma or serum, preferably from 2% to 10% of plasma or serum; or from 0.1% to 2% platelet lysate, preferably from 0.2% to 1% platelet lysate; and/or

from 5 μg/mL to 20 μg/mL of insulin, preferably from 8 μg/mL to 12 μg/mL of insulin; and/or

from 10 μg/mL to 100 μg/mL of transferrin, preferably from 30 μg/mL to 60 μg/mL of transferrin; and/or

from 10 ng/mL to 100 ng/mL of SCF, preferably from 10 ng/mL to 50 ng/mL of SCF; and/or

from 10 ng/mL to 100 ng/mL of TPO, preferably from 10 ng/mL to 50 ng/mL of TPO; and/or

from 10 ng/mL to 100 ng/mL of FLT3-L, preferably from 10 ng/mL to 50 ng/mL of FLT3-L; and/or

from 100 ng/mL to 500 ng/mL of BMP4, preferably from 150 ng/mL to 250 ng/mL of BMP4; and/or

from 50 ng/mL to 300 ng/mL of VEGF, preferably from 150 ng/mL to 250 ng/mL of VEGF; and/or

from 10 ng/mL to 100 ng/mL of IL3, preferably from 20 ng/mL to 80 ng/mL of IL3; and/or

from 10 ng/mL to 100 ng/mL of IL6, preferably from 20 ng/mL to 80 ng/mL of IL6; and/or

from 1 ng/mL to 20 ng/mL of IL1, preferably from 1 ng/mL to 10 ng/mL of IL1; and/or

from 10 ng/mL to 200 ng/mL of GCSF, preferably from 50 ng/mL to 150 ng/mL of GCSF; and/or

from 1 ng/mL to 20 ng/mL of IGF1, preferably from 1 ng/mL to 10 ng/mL of IGF1.

Preferably, the medium meets all these features.

In another particular embodiment, the liquid cell culture medium of the invention comprises (i) about 5% of plasma or serum or about 0.5% platelet lysate, and (ii) about 10 μg/mL of insulin, about 45 μg/mL of transferrin, about 22 ng/mL of SCF, about 20 ng/mL of TPO, about 300 ng/mL of FLT3-L, about 22 ng/mL of BMP4, about 200 ng/mL of VEGF, about 50 ng/mL of IL3, about 50 ng/mL of IL6, about 5 ng/mL of IL1, about 100 ng/mL of GCSF and about 50 ng/mL of IGF1.

In further particular embodiment, the liquid cell culture medium of the invention comprises (i) about 5% of plasma or serum or about 0.5% platelet lysate, and (ii) about 10 μg/mL of insulin, about 45 μg/mL of transferrin, about 24 ng/mL of SCF, about 21 ng/mL of TPO, about 21 ng/mL of FLT3-L, about 194 ng/mL of BMP4, about 200 ng/mL of VEGF, about 50 ng/mL of IL3, about 50 ng/mL of IL6, about 5 ng/mL of IL1, about 100 ng/mL of GCSF and about 5 ng/mL of IGF1.

In embodiments wherein the culture medium comprises plasma or serum, it may advantageously further comprise heparin, preferably from 0.5 U/mL to 5 U/mL heparin, more preferably from 2 U/mL to 4 U/mL heparin, and even more preferably about 3 U/mL heparin.

The present invention also relates to the use of a liquid cell culture medium of the invention for the growth and/or differentiation of cells of the hematopoietic lineage, for the differentiation of an embryoid body, for the production of hematopoietic cell graft, in particular in the absence of feeder cells.

As used herein, the term “growth” refers to the multiplication of cultured cells and the term “differentiation” refers to the acquisition by cells cultured in a culture medium of cellular characteristics committing the cells into the hematopoietic lineage. As used herein, the term “cells of the hematopoietic lineage” refers to cells to be found in the blood of mammals, in particular of humans.

The cell culture medium of the invention is particularly useful for the growth and/or differentiation of pluripotent stem cells such as embryonic stem cells and iPSC, embryoid bodies, and HSC, including early primitive HSC such as CD135+, CD110+ and/or APLNR+ HSC.

All patents, patent applications, provisional applications, and publications referred to or cited herein are incorporated by reference in their entirety, including all figures and tables, to the extent they are not inconsistent with the explicit teachings of this specification.

The following examples are given for purposes of illustration and not by way of limitation.

### EXAMPLES

### Example 1

Materials and Methods

hiPSC Amplification

The study was conducted using three different hiPSC lines: the FD136-25, reprogrammed with retroviral vectors and Thomson's combination (endogenous expression of Oct4, Sox2, Nanog and Lin28); the Pci-1426 and Pci-1432 lines (Phenocell) reprogrammed with episomes (Sox2, Oct4, KLF, cMyc). hiPSCs were maintained on CellStart (Invitrogen, Carlsbad, USA) in TESR2 medium (Stem Cell Technologies, Bergisch Gladbach, Germany) and the cells were passaged 1:6 onto freshly coated plates every 5 days using standard clump passaging with TRYple select (Invitrogen).

EB Differentiation

After 24 h, cells were transferred into differentiation medium containing 24 ng/mL of SCF, 21 ng/mL of TPO, 21 ng/mL of FLT3L, 194 ng/mL of BMP4, 200 ng/mL of VEGF, 50 ng/mL of IL3, 50 ng/mL of IL6, 5 ng/mL of IL1, 100 ng/mL of GCSF, 5 ng/mL of IGF1 (PeproTech, Neuilly-sur-Seine, France). Medium was changed every other day. EBs were dissociated on days 15, 16 and 17.

Colony Assays

At the indicated times, 1×10⁵ dissociated EB cells or 3×10⁴ cells from xenotransplanted recipient BM were plated into 3 mL of complete methylcellulose medium in the presence of SCF, IL-3, EPO and GM-CSF (PeproTech, Neuilly-sur-Seine, France). As G-CSF also stimulates mouse progenitors, it was replaced by granulocyte-macrophage colony-stimulating factor (GM-CSF). Aliquots (1 mL) of the mix were distributed into one 30 mm dish twice and maintained in a humidified chamber for 14 days. Colony-forming Cells (CFC) were scored on day 14.

Long-Term Culture-Initiating Cell Assays

Long-term culture-initiating cell (LTC-IC) assays were performed as described previously (see e.g. Miller and Eaves, Hematopoietic Stem Cell Protocols, Volume 63 of the series Methods in Molecular Medicine pp 123-141), 15-100,000 cells/well on day 17 for the EBs and on day 0 for the control CD34+. Absolute LTC-IC counts corresponded to the cell concentrations, yielding 37% negative wells using Poisson statistics.

Pseudo-Microtubules and EPC-Like Cells

For Pseudo-microtubules formation, cells were transferred onto growth factor reduced Matrigel (Corning) and culture in EGM2 medium (Lonza).

For EPC-like cells generation, cells were first plated on gelatin and cultured in EBM2 (Lonza) and split several times, after the first passaged the gelatin was no longer mandatory.

Flow Cytometry

Staining of BM cells or dissociated EBs was performed with 2×10⁵ cells in 100 μL staining buffer (PBS containing 2% FBS) with 5:100 dilution of each antibody, for 20 min at room temperature in the dark. Data acquisition was performed on a Becton Dickinson Canto II cytometer.

In Vivo Analyses of Angiogenesis Potential

1.750 10⁶ D16 single cells or hEPCs and 1.750 10⁶ hMSCs were mixed with 100 μl of Matrigel phenol red free and growth factor reduced (Corning) and subcutaneously injected into the back of nude mice (two different plugs/mouse). The controls were performed similarly but with 3.5 10⁶ hMSCs or D16 single cells or hEPCs; for each condition n=3. Two weeks later, mice were sacrificed, the matrigel plug dissected out and processed for paraffin sectioning. Sections were deparaffinized, hydrated and stained whether with Masson's trichrome, a three-color protocol comprising nuclear staining with hematoxylin, cytoplasmic staining with acid fuchsin/xykidine ponceau and collagen staining with Light Green SF (all from VWR); or whether with human Von Willebrand factor (Dako), staining were developed with histogreen substrate (Abcys) and counterstained with Fast nuclear red (DakoCytomation), dehydrated and mounted, or wether with hCD31 (R&D system) as primary antibody and donkey anti-rabbit Cy3 (Jackson ImmunoResearch) as secondary antibody and DAPI and mounted with fluoromount G.

Sorting of APLNR Positive Cells

Cells were stained with the antibody hAPJ-APC as described above. Sorting was carried out on a Moflo ASTRIOS Beckman Coulter apparatus and the purity was 98.1% APLNR positive cells.

Mouse Transplantation

NOD/SCID-LtSz-scid/scid (NOD/SCID) or NOD.Cg-Prkdc^{scid}I12rg^{tm1Wjl}/SzJ (NSG) or Foxn1−/− nude mice (Charles River, L'Abresle, France) were housed in the IRSN animal care facility. All experiments and procedures were performed in compliance with the French Ministry of Agriculture regulations for animal experimentation and approved by the local ethics committee.

Mice, 6-8 weeks old and raised under sterile conditions, were sublethally irradiated with 2.5 Grays from a 137Cs source (2.115 Gy/min) 24 h before cell injection. To ensure consistency between experiments, only male mice were used. Prior to transplantation, the mice were temporarily sedated with an intraperitoneal injection of ketamine and xylazine. Cells (0.4 ×10⁶ per mouse) were transplanted by retro-orbital injection in a volume of 100 μL using a 28.5 gauge insulin needle. A total of 140 mice were used in this study.

For the engraftment potential of the D17 cells on the three different hiPSC lines:

70 NSG mice were used as followed: 30 primary recipients, 30 secondary recipients and 10 as control.

48 NOD-SCID mice were used as followed: 20 primary recipients and 16 secondary recipients, 3 tertiary recipients and 9 as control.

For the engraftment potential of APLNR+ and APLNR− population: 10 NOD-SCID mice were used and 3 NOD-SCID as control.

In vivo assessment of the endothelial and hematopoietic potentials were probed on 9 nude mice.

Assessment of Human Cell Engraftment

Mice were sacrificed at week 12, 18 or 20. Femurs, tibias, liver, spleen and thymus were removed. Single cell suspensions were prepared by standard flushing and aliquots containing 1×10⁶ cells were stained in a total volume of 200 μL staining buffer.

Samples were stained for engraftment assessment with the following markers: hCD45 clone J33, hCD43 clone DFT1, hCD34 clone 581 (Beckman Coulter) and hCD45 clone 5B1, mCD45 clone 30F11 (Miltenyi)

The BM were pooled to allow hCD45 microbead enrichment (Miltenyi), the multilineage was assessed using the following human markers: hCD3 clone UCHT1, hCD4 clone 13B8.2, hCD8 clone B9.11, hCD14 clone RM052, hCD15 clone 80H5, hCD19 clone J3-119, hCD20 clone B9E9, hCD41clone P2, hCD61clone SZ21, hCD43 clone DFT1, hCD34-APC, hCD71 clone YDJ1.2.2 (all from Beckman Coulter antibodies, Brea, USA), CD45 clone 5B1 (Miltenyi), CD235a clone GA-R2 (Becton-Dickinson).

The blood samples were pooled to allow hCD45 microbead sorting (Miltenyi). The multilineage potential was assessed using the following human markers: hCD3 clone UCHT1, hCD4 clone 13B8.2, hCD8 clone B9.11, hCD14 clone RMO52, hCD15 clone 80H5, hCD19 clone J3-119, hCD20 clone B9E9, hCD41clone P2, hCD61clone SZ21 (all from Beckman Coulter antibodies, Brea, USA).

Non-injected mouse BM was used as a control for non-specific staining

Compensation was performed by the FMO method with anti-mouse Ig and data were acquired on a BD Canto II cytometer.

T-Cell Maturity and Functionality Assay

Blood of 3 mice was pooled to allow hCD2 microbead sorting (Miltenyi), the presence of TCR αβ and TCR γδ was assessed by flow cytometry using the following human markers: TCR αβ clone IP26A and TCR γδ clone IMMU510 (all from Beckman Coulter antibodies, Brea, USA).

Thymus and spleen cells were isolated, CFSE labelled and seeded in cell culture media complemented or not with hCD3 and hCD28 (Beckman Coulter both 1 μg/ml). After 5 days, cells were harvested and stained with anti-hCD3 clone UCHT1 and analyzed on a BD Canto II cytometer. FlowJo analysis software was used to gate on CD3⁺ T-cells and generate the overlaid histogram plots.

To assess the presence of thymocytes, thymus cells were marked with hCD1A clone BL6 (from Beckman Coulter antibodies, Brea, USA).

Assessment of the APLNR Cell Safety

Three sub-lethally irradiated NOD/SCID mice were subcutaneously injected each with 3 million APLNR positive cells. No teratoma was found after 2 months follow-up according to FDA guidelines (materials and methods).

In addition, no tumor was macroscopically detected in any mouse after analysis of the organs (140/140 mice) or after microscopic analysis of different tissues (brain, lungs, kidneys, BM, liver and gut) (140/140 mice).

Quantitative PCR

Total mRNA was isolated with the RNA minikit (Qiagen, Courtaboeuf, France). mRNA integrity was checked on a Bioanalyzer 2100 (Agilent Technologies, Massy, France). cDNAs were constructed by reverse transcription with Superscript (Life Technologies, Carlsbad, USA). PCR assays were performed using a TaqMan PCR master mix (Life Technologies) and specific primers (Applied BioSystems, Carlsbad, USA) for selected genes (see table below), together with a sequence detection system (QuantStudio™ 12K Flex Real-Time PCR System, Life Technologies). In each sample the fluorescent PCR signal of each target gene was normalized to the fluorescent signal of the housekeeping gene glyceraldehyde 3-phosphate dehydrogenase (GAPDH).

The human origin of the mRNAs from mouse BM was assessed by measuring hCD45, hCD15, hMPO, hITGA2 and hGAPDH. From CFCs post grafting and globin type expression in the mouse BM, we measured beta, gamma and epsilon globins using Taqman probes.

Controls were cultured erythroblasts generated from cord blood CD34⁺.

| Pluripotency | Hs01053049_s1 | SOX2 | Endothelial genes | | Hs00945146_m1 | TEK |
|---|---|---|---|---|---|---|
| genes | Hs00153408_m1 | MYC | | | Hs00231079_m1 | RUNX1 |
| | Hs00702808_s1 | LIN28A | | | Hs00911700_m1 | KDR |
| | Hs04260366_g1 | NANOG | | | Hs01574659_m1 | NOS3 |
| | Hs00358836_m1 | KLF4 | | | | |
| | Hs00742896_s1 | POU5F1 | Human housekeeping | | Hs00357333_g1 | Human beta |
| Mouse | Mm99999915_g1 | Murine | genes | | | actin |
| housekeeping | | GAPDH | | | Hs02758991_g1 | Human |
| gene | | | | | | GAPDH |
| Hematopoietic | Hs00924296_m1 | MPO | Hs01076122_m1 | DNTT | Hs00269972_s1 | CEBPA |
| genes | Hs01106466_s1 | FUT4 | Hs00172743_m1 | RORC | Hs01115556_m1 | MITF |
| | Hs00174029_m1 | cKIT | Hs00962186_m1 | CD3G | Hs01029175_m1 | NFIB |
| | Hs01116228_m1 | ITGA2B | Hs00169777_m1 | PECAM | Hs04188695_m1 | HOPX |
| | Hs00766613_m1 | APLNR | Hs00231119_m1 | GATA2 | Hs00171406_m1 | HLF |
| | Hs00161700_m1 | STIL | Hs00959427_m1 | EPOR | Hs01070488_m1 | RBPMS |
| | Hs00231119_m1 | GATA2 | Hs00610592_m1 | KLF1 | Hs00171569_m1 | HMGA2 |
| | Hs00995536_m1 | BMI1 | Hs01085823_m1 | GATA1 | Hs00223161_m1 | PRDM16 |
| | Hs00941830_m1 | NCAM1 | Hs04186042_m1 | RUNX1 | Hs01017441_m1 | MEIS1 |
| | Hs04189704_m1 | PTPRC | Hs00176738_m1 | MATK | Hs00414553_g1 | NKX2-3 |
| | Hs00174333_m1 | CD19 | Hs00180489_m1 | MPL | Hs00971097_m1 | MLLT3 |
| | Hs00162150_m1 | SPIB | Hs04334142_m1 | FLI1 | Hs00925052_m1 | GATA3 |
| | Hs00958474_m1 | IKZF1 | Hs00268388_s1 | SOX4 | Hs01128710_m1 | IRF8 |
| | Hs01851142_s1 | RAG2 | Hs00193527_m1 | C-MYB | Hs00256884_m1 | HOXB4 |
| | Hs00266821_m1 | HOXA9 | Hs01554629_m1 | ERG | Hs00610592_m1 | KLF1 |
| | Hs00931969_m1 | RORA | Hs01547250_m1 | LEF1 | Hs04334142_m1 | FLI1 |
| | Hs00959427_m1 | EPOR | Hs00176738_m1 | MATK | Hs00180489_m1 | MPL |

Statistical Analysis

All statistics were determined with R Software 3.1.1 (2014-07-10) (R Core Team, 2013), INGENUITY and SAM Software. Data are represented with hierarchical clustering and PCA.

Results

The first transplantable HSCs are produced during embryonic development from a specialized population of endothelial cells (ECs) called the hemogenic endothelium. Following endothelial-to-hematopoietic transition (EHT), these hemogenic ECs differentiate into hematopoietic cells (HCs) including HSCs, enter the circulation, amplify in the fetal liver, and attain the BM, their definitive site of residence. These early steps of developmental hematopoiesis are fully recapitulated in embryoid body (EB) cultures, notably, the generation of hemogenic ECs and the budding of HCs.

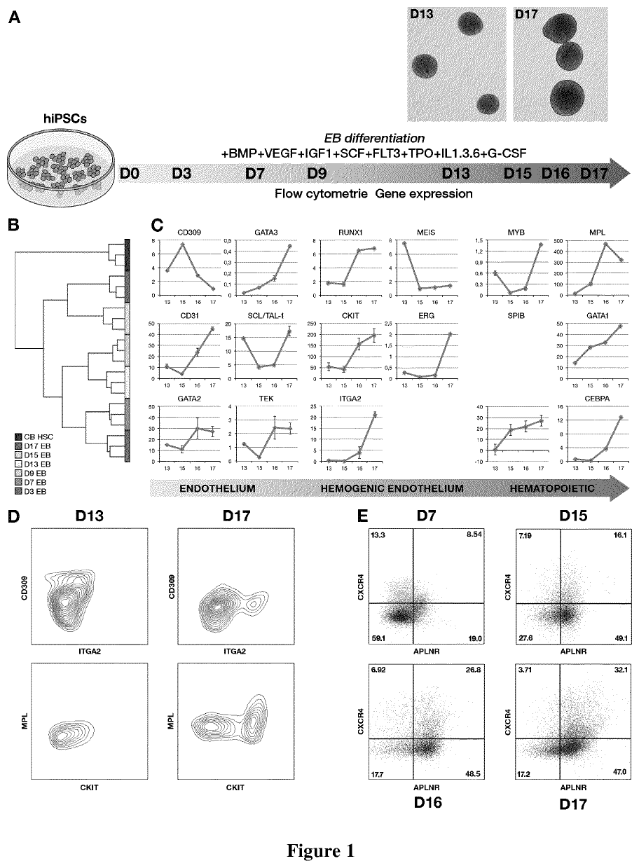

The inventors developed a one step, vector-free and stromal-free system procedure to direct differentiation of hiPSCs into the endo-hematopoietic lineage. All the cytokines and growth factors are present from Day (D) 0 to the end of the culture period, to fulfill any need. Many studies use a 14-D long protocol and isolate the cells between D11 and 14 based on the presence of hematopoietic bursts on EBs. The inventors did not obtain burst even at D17 therefore assessing a dramatic delay in the differentiation process (FIG. 1A). These culture conditions were applied to three different hiPSCs cell lines differing by their reprogramming protocols e.g. episomal or retroviral, with similar differentiation efficacies hence demonstrating the sturdiness of the method.

Aiming to determine the point of hemogenic EC/early HC commitment, the inventors analyzed EB cells by qRT-PCR on D3, 7, 9, 13, 15, 16 and 17 for the expression of pluripotency genes and for 49 of the key endothelial- and hematopoietic-specific genes, taking as reference the molecular profiles of CD34⁺ cord blood HSCs. Hierarchical clustering analysis (FIG. 1B) indicated the presence of two main groups, one associated with CD34⁺ cord blood cells and another one associated with EB cells (D3 to day 17). The latter is divided into two distinct clusters: one comprising the early EB cells (D3 to 13) and another one encompassing the late EB cells (D15 to 17) suggesting the existence of a balance point between D13 and 15. More in depth analysis of the qPCR patterns identified D13 as the point of EC commitment on the basis of CD309 (VEGFR2) mRNA expression and D16 as the point of putative hemogenic endothelial commitment on the basis of RUNX1 mRNA expression (FIG. 1C). Hematopoietic-specific markers were also up-regulated from D16 such as ITGA2 (Integrin alpha-2) and CEBPA (CCAAT enhancer binding protein alpha) in keeping with the onset of RUNX1 expression. D17 cells exhibited a tendency to converge towards the CD34⁺ cell profile as shown by an increased expression of the HC-specific genes (data not shown). To confirm the balance point, the inventors analyzed the cell population by flow cytometry for surface expression of CD309 as EC marker and MPL, CKIT and ITGA2 as early HC markers. Flow cytometry analysis confirmed the decrease of CD309 from D13 to 17 and the increase of ITGA2, CKIT and MPL (FIG. 1D) in keeping with the q-PCR analysis (FIG. 1C). They further identified a cell population displaying the expression of the APELIN receptor (APLNR) related to early hematopoietic commitment on D15 to 17 cells and, within, a sub-fraction that progressively acquired the expression of the locomotion and homing receptor CXCR4 (FIG. 1E).

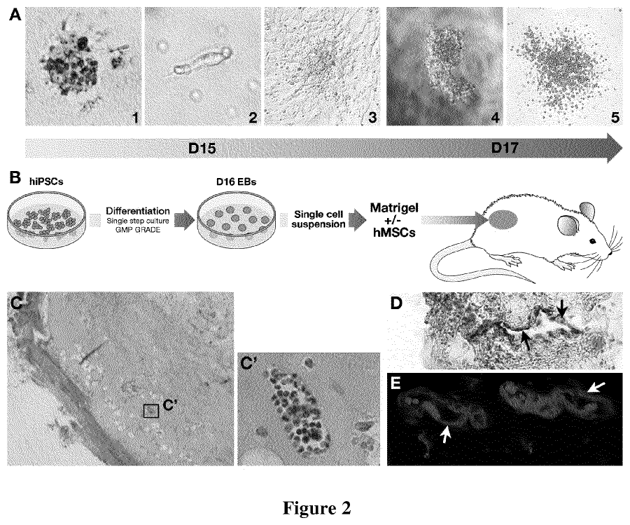

They next evaluated the endothelial and hematopoietic potential of EB cells on D15, 16 and 17 using dedicated in vitro functional tests (FIG. 2A). D15 cells displayed strong endothelial-forming potential as revealed by their capacity to generate endothelial colony-forming cells (CFC-EC) (FIG. 2A1), pseudo-microtubules (FIG. 2A2) and EC-like cells (FIG. 2A3), but lacked hematopoietic-forming capacity, being unable to generate clonogenic colonies and displaying a very low frequency of long term culture-initiating cells. In contrast, D17 cells lacked endothelial potential but exhibited a significant increase in hematopoietic capacity (FIG. 2A4, A5), confirming the onset of hematopoietic commitment within this period.

The D16 balance point was probed in vivo by transplanting the cells subcutaneously in a Matrigel (growth factor reduced) plug with or without human Mesenchymal Stem Cells (hMSCs) into immunocompromised Foxn1−/− (nude) mice (FIG. 2B). Two weeks following transplantation, human vascular structures (FIG. 2C, D), made of human CD31⁺ cells and von Willebrand cells were detected in the graft containing D16 cells and (/) hMSCs. QRT PCR revealed the expression of hVEGFR2, hENG (ENDOGLIN), hPECAM-1 in the grafts made with D16 cells/hMSCs and, as expected, in the grafts made with endothelial progenitor cells/hMSCs (data not shown). Moreover, D16 cells/hMSCs plugs expressed human beta, gamma and epsilon globin transcripts, while D16 cells alone plugs expressed only human epsilon globin transcript disclosing a block of maturation. D16 cells thus displayed a balanced endothelial-hematopoietic pattern in keeping with in vitro results.

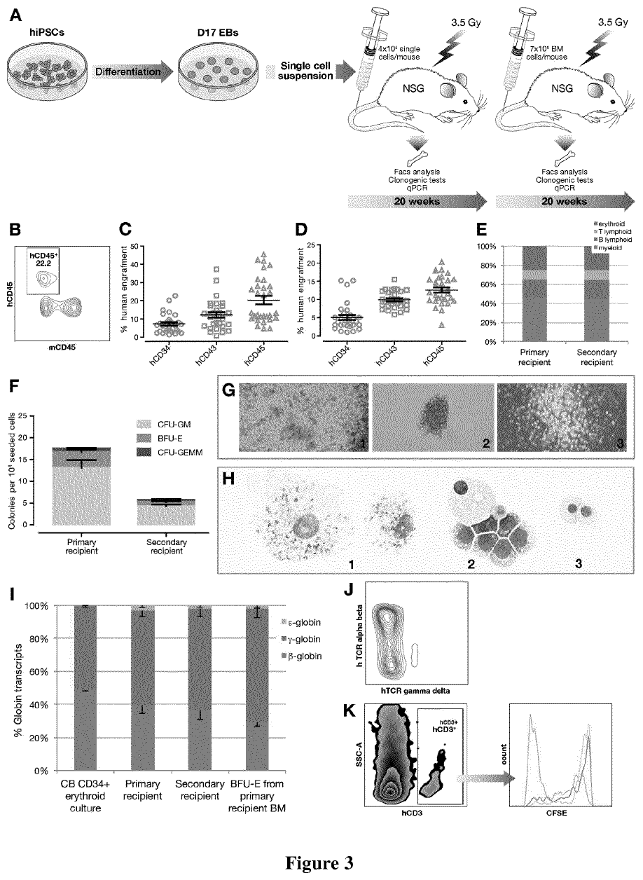

Since D17 cells displayed the strongest hematopoietic capability, the inventors transplanted 4×10⁵ cells into sublethally irradiated (3.5 gray) 8-week-old immunocompromised mice over a period of 20 weeks followed by a challenging secondary transplantation in a similarly-treated immunocompromised recipient over an additional period of 20 weeks (FIG. 3A). The presence of human HCs was quantified through their surface expression of hCD34, hCD43 and hCD45 (FIG. 3B, C, D). Multilineage human hematopoiesis was evident in the 30/30 primary recipient mice (FIG. 3C), with a mean of 20.3+/−2.9% hCD45⁺ cells among total mouse BM mononucleated cells, i.e., 203 times the threshold of 0.1% usually considered as positive for human HC engraftment in NSG mice (Tourino et al., The hematology journal: the official journal of the European Haematology Association/EHA 2, 108-116 (2001)), and 12.2+/−1.5% hCD43⁺ and 7.29+/−1.0% hCD34⁺ (FIG. 3C). Within the hCD45⁺ BM population, several human HC lineages were detected including B cells (CD19⁺CD45⁺), T cells (CD3⁺CD45⁺, CD4⁺CD45⁺), macrophages (CD14⁺CD45⁺, CD15⁺CD45⁺) (FIG. 3E, FIG. S3H) and erythroid progenitors/precursors (CD235a⁺CD45⁺) (not shown). Sorted hCD45⁺ peripheral blood cells displayed the same multilineage pattern indicating a peripheralization of the grafted cells. The human origin of the engrafted cells was confirmed (n=30/30) by q-PCR using human-specific primers for CD45, CD15, MPO, ITGA2 and GAPDH genes. A human-specific clonogenic assay performed on BM cells isolated from the first recipient mice revealed an overall frequency of 17.5+/−4.3 clones out of 10⁴ total BM cells (FIG. 3F) distributed into CFU-GEMM, BFU-E and CFU-GM colonies (FIG. 3G1, 2, 3). Cytospin analysis revealed the presence of mature macrophages, histiomonocytes, myelocytes and erythroblasts (FIG. 3H1, 2, 3). 7.10⁶ BM cells of the primary recipient were challenged in a secondary (n=30) recipient (FIG. 3B, D) and eventually a tertiary recipient (n=3) in the case of NOD-SCID mice (data not shown). Human CD45⁺ cells represented 12.6+/−3.9 of the mononucleated BM cells (FIG. 3B, D), indicating a sustained reconstitution capacity. Multilineage engraftment was found in 30/30 mice (FIG. 3E). The overall cloning efficiency of human CFCs was 5.5+/−3.1% in 10⁴ total mouse BM cells, pointing to a robust and prolonged self-renewal capacity (FIG. 3F-H). The human origin of the engrafted cells was confirmed as above.

To ensure the functionality of the grafted cells, the inventors analyzed the ability of the human erythroid precursors, from mouse bone marrows, to undergo hemoglobin switching in vivo and tested the phenotype and the functionality of T cells. Engrafted cells from both primary and secondary recipients were able to generate human erythroid progenitors displaying high amounts of β (respectively 39.51+/−4.95 and 36.61+/−5.86) and γ globin (respectively 57.49+/−3.95 and 61.39+/−4.86) while ε globin was dramatically reduced to respectively 3.0+/−1.2% and 2.1+/−1.1% of total globin (FIG. 3I). Silencing embryonic and activating adult globin expression are hallmarks of definitive erythrocytes

Peripheral blood-isolated hCD2⁺ T cells displayed high amounts of TCRαβ (FIG. 3J) and very low amounts of TCRγδ assessing human T cells ability to mature. Thymus and spleen cells were tested on their in vitro ability to expand, as measured by CSFE-labeling, under hCD3 and hCD28 stimulation. After 5 days, thymus (FIG. 3K) and spleen (data not shown) cells gated on hCD3⁺ expression showed a high capacity to expand thereby demonstrating the functionality of human T cells.

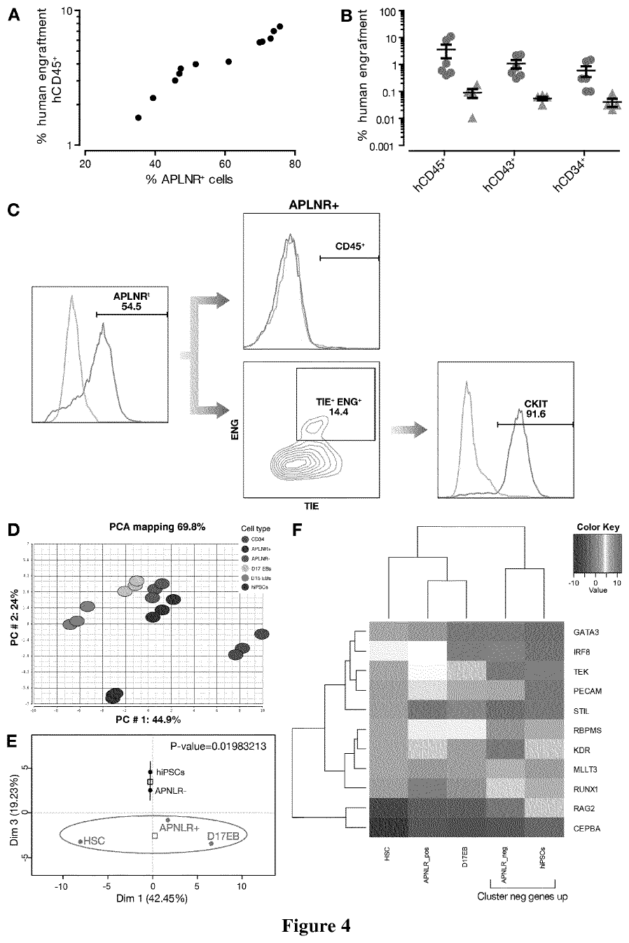

FIG. 4A illustrates the percentage of APLNR⁺ cells in EBs at culture incipience reported to the percentage of hCD45⁺ cells in the primary NOD-SCID recipient 18 weeks after grafting. The inventors sorted the APLNR⁺ and − populations and compared their engraftment capacities in vivo in the NOD-SCID model. APLNR⁺ cells successfully reconstituted hematopoiesis after 18 weeks (FIG. 4B). Human CD45⁺ cells represented 6.6+/−1.9% of the mononucleated cells in mouse BM, 3.4+/−2.5% were hCD43⁺ and 1.1+/−0.4% were hCD34⁺ in 6/6 grafted mice (FIG. 4B, FIG. 5). Flow cytometry analysis of the BM cells revealed a human multilineage phenotype (data not shown). D17 APLNR⁺ cells did not harbor any CD45⁺ cell therefore indicating that the reconstitution capacity was not due to the presence of hCD45⁺ committed progenitors (FIG. 4C). In contrast, APLNR⁻ cells failed to engraft at a significant level in 4/4 mice with 0.08+/−0.01% hCD45⁺ cells in mouse BM (FIGS. 4B and 5). Of interest, the APLNR⁺ fraction exhibited a homogeneous population of ENG⁺/TIE⁺/CKIT⁺ (FIG. 4C) described in mice to enhance definitive hematopoiesis.

To further characterize the APNLR⁺ population, the inventors compared the molecular profiles of APLNR⁺ and − cells to that of hiPSCs and to control CD34⁺ HSCs with respect to the expression of gene sets representative to the pluripotent state and to endothelial, hemogenic endothelial or hematopoietic commitment. Principal component analysis (PCA) (FIG. 4D) of the gene expression levels as expressed by the ΔCt with the set of 49 mRNAs studied as variables and the six cell populations as observations revealed that the first component, likely corresponded to the factor “hematopoietic differentiation”, accounted for 44.9% of the variance. Aiming to further reveal the traits involved in the grafting potential, they compared by PCA the APLNR⁻, D17 and HSC population to the APLNR⁻ and hiPSC population. The third component that accounted for 19.23% of the variance segregated the population into two groups differing by their grafting potential (FIG. 4E). A statistical SAM test which measures the strength of the relationship between gene expression and a response variable pointed out 8 genes (FDR<10%) which are significantly more up-regulated in the group unable of graft, among them endothelial genes as TEK, PECAM, and KDR (FIG. 4F).

On the basis of these findings, the inventors showed that the generation of long-term multipotent HSCs supporting multi-lineage hematopoietic reconstitution and self-renewal in vivo, passes through an early differentiated cell undergoing EHT and expressing APLNR. These experiments have been performed under GMP-grade culture conditions, thereby opening the way to the use of pluripotent stem cells as a prioritized source of cells for HSC transplantation.

### Example 2

Materials and Methods

hiPSC amplification, EB differentiation, assessment of human cell engraftment, T cell maturity and functionality assay, quantitative PCR were performed as described above.

Cell Sorting

Dissociated EB cells were stained with the antibody CD110-PE (MPL) or CD135-PE (FLT3) then re-stained with PE-MicroBeads (Miltenyi) and finally sorted with the MACS® cell separation device.

Mouse Transplantation

NOD.Cg-Prkdc^{scid}I12rg^{tm1Wjl}/SzJ (NSG) (Charles River, L'Abresle, France) were housed in the IRSN animal care facility. All experiments and procedures were performed in compliance with the French Ministry of Agriculture regulations for animal experimentation and approved by the local ethics committee.

Mice, 6-8 weeks old and raised under sterile conditions, were sublethally irradiated with 3.5 Grays from a 137Cs source (2.115 Gy/min) 24 h before cell injection. To ensure consistency between experiments, only male mice were used. Prior to transplantation, the mice were temporarily sedated with an intraperitoneal injection of ketamine and xylazine. MPL+ or FLT3+ cells (10⁴ per mouse) were transplanted by retro-orbital injection in a volume of 100 μL using a 28.5 gauge insulin needle.

For the engraftment potential of the D17 cells on the three different hiPSC lines:

50 NSG mice were used as followed: 25 primary recipients, 25 secondary recipients.

Bioinformatics

Bioinformatics analysis were performed in R environment software version 3.0.2. Public available transcriptome datasets were downloaded as normalized matrix (GSE format: gene expression matrix) on database Gene Expression Omnibus (GEO) (http://www.ncbi.nlm.nih.gov/geo/).

Results

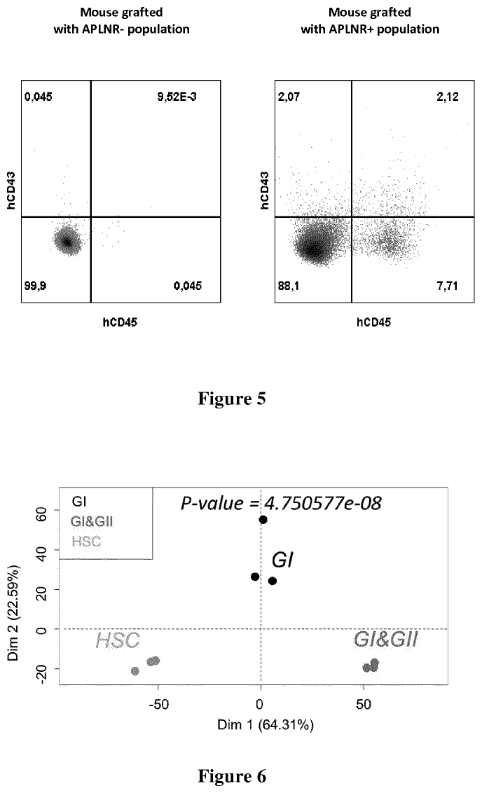

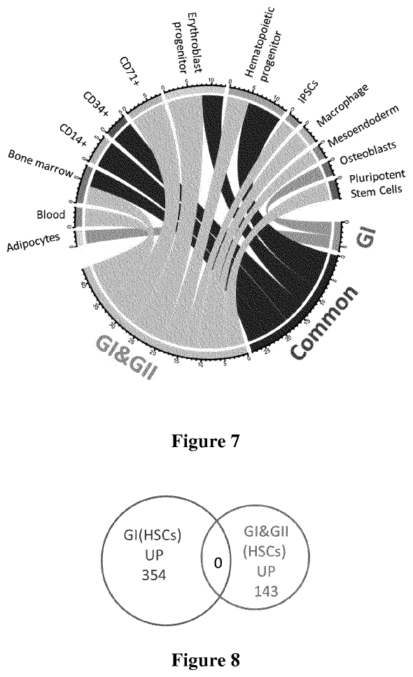

In order to better characterized scid repopulating cells (SRCs) coming from differentiation of pluripotent cells (IPSCs: induced pluripotent cells), the inventors analyzed transcriptome samples taking account to their xenotransplantation capacities to performed primary or secondary transplant success. Transcriptome series were merged in order to build a control group of sorting hematopoietic stem cells (HSC, with phenotype: CD34+CD38−CD90+, n=3) compared to a group of IPSCs with only primary xeno-transplantation capacities (group GI, n=3) and to a group of IPSCs with primary and secondary xeno-transplant capacities (group GI&GII, n=3). After mathematical correction of batch-effect, one way ANOVA (analysis of variance, p-value less than 1E-4) supervised analysis was performed between the 3 defined sample groups (HSCs, GI, GI&II). Unsupervised principal component analysis performed on 5859 differential genes between the groups allowed to significantly discriminated sample groups with a p-value of 4.75E-8 on the principal map (FIG. 6). These results suggest that selected genes are potentially relevant to study the xeno-transplant phenotype of SRC-IPSCs taking in account their capacities to give primary and/or secondary transplant. Moreover, batch-effect related to transcriptome datasets showed no influence on phenotype group discrimination during this unsupervised analysis. Supervised analysis by Significance analysis for microarray (SAM) between each xeno-transplant group and HSC group was performed in order to found HSCs biomarkers in each group of SRCs-IPSCs. On relational circosplot (FIG. 7), a greater diversity of HSCs biomarkers was found for GI&GII group than for GI group. This specific diversity for GI&GII group comprised functional categories such as: mesoderm, pluripotent stem cell and IPSCs. The specificity of biomarker for GI group concerned more mesenchymal phenotype such as osteoblast and adipocyte. In other way, common biomarkers were more found in hematopoietic lineage such as: hematopoietic progenitor, erythroblast progenitor, CD34+ cells, bone marrow and CD14+ cells. In order to see the influence of HSC expression profile (CD34+38−90+) in the characterization of SRC-IPSCs, HSC group was introduced in supervised analysis to compare the SRC-IPSCs groups. Expression heatmaps performed by unsupervised classification showed that each xeno-transplant SRC-IPSCs group expressed some HSC related biomarkers: HSCs biomarkers in relation with GI group of SRC-IPSCs, HSCs biomarkers in relation with GI&GII group of SRC-IPSCs (data not shown). Venn diagram which compared HSCs biomarkers enriched in each group of SRC-IPSCs showed any gene in common (FIG. 8). SRC-IPSCs cell which have GI&GII capacities specifically expressed some cell surface molecules as compared to GI group: FLT3 (CD135) and MPL (CD110).

Based on this in silico study, the inventors decided to study more specifically MPL and FLT3 receptors for which antibodies allowing their immunomagnetic screening are available.

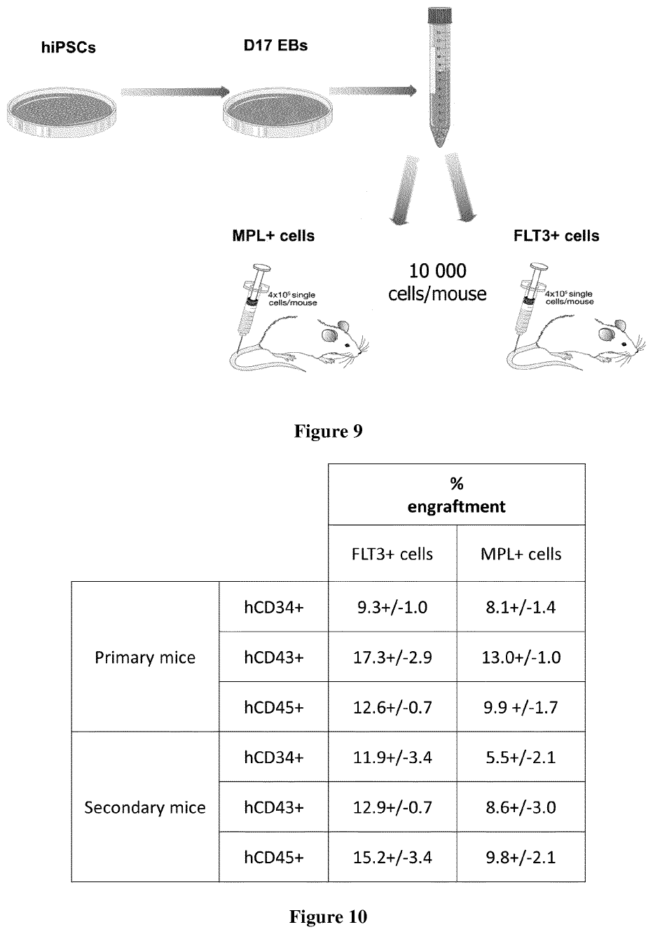

After 17 days of hiPSCs differentiation in EBs in an appropriate medium (see example 1), the inventors carried out the screenings and then grafted 10.000 cells/NSG immunosupressed mouse (n=15 for FLT3 and n=15 for MPL) (FIG. 9). Twenty weeks later, mice were sacrificed and their bone marrows, spleens, livers and thymus as well as blood samples were studied.

For both populations (FLT3+ and MPL+ cells), a high level of engraftment was obtained (12.6+/−0.7% of hCD45+ for the FLT3+ population and 9.9+/−1.7% for the MPL+ population) (FIG. 10) and human cells from all the hematopoietic lineages were found. Human red blood cells was found to produce β-globin, blood circulating T lymphocytes expressed TCRγδ at their surface and lymphocytes from thymus or spleen were capable of in vitro proliferation after activation, underlying that FLT3+ or MPL+ cells are capable of definitive hematopoiesis.

For each primary mouse, seven millions of bone marrow cells were grafted in secondary mice. After 20 weeks, these secondary mice were sacrificed and analyzed as described above.

All the secondary mice exhibited a high level of engraftment (15.2+/−3.4% of hCD45+ for the FLT3+ population and 9.8+/−2.1% for the MPL+ population) (FIG. 10), a proven multilineage, and human grafted cells were capable of definitive hematopoiesis.

Therefore, the cells obtained through hiPSCs differentiation according to the inventor's protocol and that express FLT3 or MPL at their surface are capable of long-term multilineage engraftment and self-renewal in vivo.

1-20. (canceled)

21. An in vitro method of preparing hematopoietic cell graft or enriching a population of cells for hematopoietic stem cells that are capable of long-term multilineage engraftment and self-renewal, said method comprising
a) providing a population of cells comprising hematopoietic stem cells, and
b) sorting cells of said population based on the expression of cell surface antigens CD135 and/or CD110, and
c) recovering cells that are CD135+ and/or CD110+.

22. The method according to claim 21, wherein in step b) cells are sorted based on the expression of cell surface antigen CD110, and in step c) recovered cells are CD110+.

23. The method according to claim 22, wherein in step b) cells are further sorted based on the expression of cell surface antigen CD135, and in step c) recovered cells are CD110+ CD135+.

24. The method according to claim 21, further comprising, before, after or simultaneously to step b), sorting cells based on the expression of the apelin receptor (APLNR) and recovering cells that are APLNR+.

25. The method according to claim 21, wherein the population of cells provided in step a) comprises hematopoietic stem cells obtained from peripheral blood, placental blood, umbilical cord blood, bone marrow, liver and/or spleen and/or comprises immortalized hematopoietic stem cells.

26. The method according to claim 21, wherein the population of cells provided in step a) comprises hematopoietic stem cells obtained from in vitro differentiation of pluripotent stem cells, induced pluripotent stem cells or embryonic stem cells.

27. The method according to claim 26, wherein the method further comprises, before step a),
providing pluripotent stem cells or induced pluripotent stem cells,
inducing embryoid body (EBs) formation,
culturing EBs in a liquid culture medium triggering differentiation of the pluripotent stem cells into the endo-hematopoeitic lineage, and
dissociating EB cells,
thereby obtaining the population of cells provided in step a).

28. The method according to claim 27, wherein the liquid culture medium comprises stem cell factor (SCF), thrombopoietin (TPO), FMS-like tyrosine kinase 3 (FLT3) ligand, bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

29. The method according to claim 28, wherein the liquid culture medium comprises (i) plasma, serum, platelet lysate and/or serum albumin, and (ii) transferrin or a substitute thereof, insulin or a substitute thereof, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

30. The method according to claim 27, wherein the pluripotent stem cells are cultured in the liquid culture medium for 14 to 19 days, for 15 to 18 days, or for 17 days.

31. A hematopoietic cell graft comprising:
a) hematopoietic cells and a pharmaceutically acceptable carrier, wherein at least 10% of cells are CD135+ and/or CD110+ hematopoietic stem cells; or
b) hematopoietic cells and a pharmaceutically acceptable carrier, wherein at least 10% of cells are CD135+ and/or CD110+ hematopoietic stem cells and at least 10% of cells are CD110+ hematopoietic stem cells.

32. A hematopoietic cell graft prepared according to the method of claim 21.

33. A method of treating a disease selected from multiple myeloma, non-Hodgkin's lymphoma, Hodgkin's disease, acute myeloid leukemia, acute lymphoblastic leukemia, chronic myeloid leukemia, myelodysplastic syndromes, myeloproliferative disorders, chronic lymphocytic leukemia, juvenile chronic myeloid leukemia, neuroblastoma, ovarian cancer, germ-cell tumors, autoimmune disorders, amyloidosis, aplastic anemia, paroxysmal nocturnal hemoglobinuria, Fanconi's anemia, Blackfan-Diamond anemia, thalassemia major, sickle cell anemia, severe combined immunodeficiency, Wiskott-Aldrich syndrome and inborn errors of metabolism comprising administering a hematopoietic stem cell graft according to claim 30 to a subject in need of treatment.

34. The method according to claim 33, wherein said graft is an autologous, syngeneic or allogeneic transplantation.

35. A liquid cell culture medium comprising (i) plasma, serum, platelet lysate and/or serum albumin, and (ii) transferrin or a substitute thereof, insulin or a substitute thereof, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

36. The liquid cell culture medium according to claim 35, comprising (i) plasma, serum and/or platelet lysate, and (ii) transferrin, insulin, stem cell factor (SCF), thrombopoietin (TPO), FMS-like tyrosine kinase 3 ligand (FLT3-L), bone morphogenetic protein 4 (BMP4), vascular endothelial growth factor (VEGF), interleukin 3 (IL3), interleukin 6 (IL6), interleukin 1 (IL1), granulocyte-colony stimulating factor (GCSF) and insulin-like growth factor 1 (IGF1).

37. The liquid cell culture medium according to claim 35 comprising:
from 10 to 100 ng/mL of SCF; and/or
from 10 to 100 ng/mL of TPO; and/or
from 10 to 100 ng/mL of FLT3-L; and/or
from 50 to 300 ng/mL of BMP4; and/or
from 50 to 300 ng/mL of VEGF; and/or
from 10 to 100 ng/mL of IL3; and/or
from 10 to 100 ng/mL of IL6; and/or
from 1 to 20 ng/mL of IL1; and/or
from 10 to 200 ng/mL of GCSF; and/or
from 1 to 20 ng/mL of IGF1.

38. The liquid cell culture medium according to claim 35 comprising:
from 1% to 20% of plasma or serum; or from 0.1% to 2% platelet lysate; or from 0.1% to 2% serum albumin; and/or
from 5 μg/mL to 20 μg/mL of insulin or a substitute thereof; and/or
from 10 μg/mL to 100 μg/mL of transferrin or a substitute thereof.

39. A method for the growth and/or differentiation of cells of the hematopoietic lineage, for the differentiation of an embryoid body, and/or for the production of hematopoietic cell graft comprising culturing hematopoietic stem cells in a culture medium according to claim 35.

40. A method for treating disorders related to deficiencies in hematopoiesis caused by disease or myeloablative treatments comprising administering a hematopoietic cell graft according to claim 31 to a subject in need of treatment.
